(19) United States
(12) Patent Application Publication
(10) Pub. No.: US 2006/0116321 A1
(43) Pub. Date: Jun. 1, 2006

(54) Immunosuppressive exosomes

(76) Inventors: Paul D. Robbins, Mt. Lebanon, PA (US); Seon-Hee Kim, Pittsburgh, PA (US); Peter Wehling, Dusseldorf (DE)

Correspondence Address: BAKER & BOTTS, 30 ROCKEFELLER PLAZA, NEW YORK, NY, 10112, US

(21) Appl. No.: 11/173,339

(22) Filed: Jul. 1, 2005

Related U.S. Application Data
(60) Provisional application No. 60/585,302, filed on Jul. 1, 2004.

(51) Int. Cl. A61K 38/20; A61K 38/17

(52) U.S. Cl. 514/12; 424/85.2

(57) ABSTRACT

The present invention relates to methods and compositions for use in mediating an immunosuppressive reaction. The compositions of the invention comprise exosomes having immunosuppressive activity. Such exosomes may be derived from a variety of different cell types, including antigen presenting cells such as dendritic cells and macrophages. Prior to isolation of exosomes, the cells may be genetically engineered to express molecules capable of enhancing the immunosuppressive activity of said exosomes and/or may be exposed to one or more agents, such as cytokines or cytokine inhibitors, which are also capable of enhancing the immunosuppressive activity of exosomes. The present invention also relates to the use of such exosomes for the treatment of diseases and disorders associated with undesirable activation of the immune system. The present invention also includes exosomes isolated directly from serum that have been shown to be immunosuppressive.

### PRIORITY CLAIM

This application claims priority to U.S. Provisional Application No. 60/585,302, filed on Jul. 1, 2004, which is incorporated by reference herein in its entirety.

### 1. INTRODUCTION

The present invention relates to methods and compositions for use in mediating an immunosuppressive reaction. The compositions of the invention comprise exosomes having immunosuppressive activity. Such exosomes may be derived from a variety of different cell types, including antigen presenting cells such as dendritic cells and macrophages. Prior to isolation of exosomes, the cells may be genetically engineered to express molecules capable of enhancing the immunosuppressive activity of said exosomes and/or may be exposed to one or more agents, such as cytokines or cytokine inhibitors, which are also capable of enhancing the immunosuppressive activity of exosomes. The present invention also relates to the use of such exosomes for the treatment of diseases and disorders associated with undesirable activation of the immune system. The present invention also includes exosomes isolated directly from serum that have been shown to be immunosuppressive.

### 2. BACKGROUND OF THE INVENTION

Autoimmune disorders are characterized by the loss of tolerance against self-antigens, activation of lymphocytes reactive against “self” antigens (autoantigens), and pathological damage in target organs. Autoimmune disorders include rheumatoid arthritis, osteoarthritis, allergies, systemic lupus erythematosus, autoimmune disease type 1 diabetes, inflammatory disorders, asthma, etc. In most situations, autoimmunity may be prevented by peripheral tolerance, which is a process presumably involving a series of multi-step interactions between antigen presenting cells (APC), in particular dendritic cells (DC), and effector T cells.

Rheumatoid arthritis (RA), for example, is a debilitating, systemic autoimmune disease characterized by chronic inflammation of the distal diarthriodial joints. Once RA is established, the affected joints exhibit inflammatory cell infiltration and synovial hyperplasia that contribute to the progressive degradation of cartilage and bone, resulting in the complete loss of normal joint function. Recently, biological agents that modulate the pro-inflammatory activities of TNF-α and IL-1β have shown efficacy as novel anti-arthritic drugs (Evans and Robbins, J. Rheumatol. 21:779-782 (1994); Robbins and Evans, Gene Ther. 3:187-189 (1996); Evans and Robbins, Curr Opin Rheumatol. 8:230-234 (1996); Evans et al., Arthritis Rheum. 42:1-16 (1999); Ghivizzani et al., Clin Orthop. 379 (Suppl):S288-299 (2000)).

In addition, gene transfer of a variety of therapeutic agents has been shown to be effective in animal models of arthritis. In particular, local intra-articular and systemic injection of adenoviral vectors expressing a variety of therapeutic agents such as sTNF-alpha receptor, IL-1Ra, sIL-1 receptor Type I and Type II, IL-10, vIL-10 and IL-4 was able to confer a significant anti-arthritic effects in mouse, rat, and rabbit models of arthritis (Arend, Lancet. 341: 155-156 (1993); Bandara et al., Proc Natl Acad Sci USA. 90:10764-10768 (1993); Ghivizzani et al., Proc Natl Acad Sci USA. 95: 4613-4618 (1998); Mori et al., J. Immunol. 157:3178-3182 (1996); Joosten et al., Arthritis Rheum. 39:797-809 (1996); Kim et al., Arthritis Res. 2:293-302; Kim et al., J. Immunol. 164: 1576-1581 (2000)).

Interestingly, local delivery of these therapeutic agents to one joint or paw by gene transfer resulted in a therapeutic effect in the contralateral knee or in untreated paws. For example, intra-articular injection of an adenoviral vector expressing vIL-10, the Epstein Barr Virus encoded IL-10 gene, resulted in a reduction in disease pathology, reducing the white blood cell infiltrate and improving cartilage metabolism in not only the injected knee, but also in the contralateral control knee (Lechman et al., J. Immunol. 163:2202-2208 (1999)). This effect was termed the contralateral effect due to the initial observation made in a rabbit knee model of arthritis. A similar effect has been observed with injection of retroviral vectors, liposomes and even with genetically modified synovial fibroblasts into rabbit, rat and murine joints (Ghivizzani et al., Gene Ther. 4:977-982 (1997); Ceponis et al., Arthritis Rheum. 44:1908-1916 (2001); Kim et al. Mol Ther. 6:591-600 (2002)).

Recent analysis of the effect has suggested that modification of function of antigen presenting cells (APCs), such as macrophages and dendritic cells (DCs), play a major role in conferring the antigen-specific effects to distal joints (Whalen et al., Mol Ther. 4:543-550 (2001); Kim et al., J. Immunol. 166:3499-3505 (2001)). In particular, the therapeutic role of DC has been further evidenced by the demonstration that bone marrow derived DC, genetically modified in culture, are effective agents to reverse established arthritis in murine models. For example, gene transfer of IL-4 or FasL to DC followed by injection into mice with established arthritis resulted in a significant regression of arthritis, more than half of the treated mice becoming disease free for at least two months post-treatment (Kim et al., J. Immunol. 166:3499-3505 (2001); Kim et al., Mol Ther. 6:584-590 (2002); Morita et al., J. Clin Invest. 107:1275-84).

Dendritic cells are professional APCs that play a crucial role in controlling immune responses, and can either augment or reduce autoimmune responses by a variety of mechanisms. DCs genetically engineered to express immunosuppressive molecules have also been considered as an attractive approach to alleviating foreign graft rejection and autoimmune disorders (Lu et al., 1999, J. Leukoc. Biol. 66:293-296). For example, delivery of cytotoxic T lymphocyte antigen 4-immunoglobulin (CTLA4Ig) into DCs by a recombinant adenovirus (Ad) vector has been shown to promote tolerogenicity and survival of these DCs in allogeneic recipients (Lu et al., 1999, Gene Ther. 6:554-563).

However, there are potential problems associated with such an approach. For example, although tolerogenicity may be enhanced in a host by the administration of immature DCs that are hyporesponsive, transduction of DCs by a virus-based vector may stimulate maturation, leading to enhanced immunostimulatory capacity (Rea et al., 1999, J. Virol. 73:10245-10253). Thus, despite their potentially therapeutic effect in slowing disease onset, administration of whole dendritic cells may have undesirable consequences.

The release of small lipid vesicles, termed exosomes, by a variety of cells types, including DCs, has been well documented for over 30 years. Exosomes are small particles of 30 to 100 nM in size that were first described as small particles, containing 5′ nucleotidase activity and transferrin receptor, derived from the late endosomal compartment and released from tumor lines (Culvenor et al., J. Cell Biochem. 20:127-138 (1982)) and from reticulocytes (Johnstone et al. J. Biol. Chem. 262:9412-9420 (1987)). Exosomes are generated by inward or reverse budding, resulting in particles that contain cytosol and exposed extracellular domains of certain membrane-associated proteins (Stoorvogel et al., Traffic 3:321-330 (2002)). Exosomes have been shown to be distinct from apoptotic bodies as well as from larger microvesicles that appear to be generated by plasma membrane shedding. Many cell types have been shown to generate exosomes including dendritic cells, reticulocytes, T lymphocytes, B cells, platelets, epithelial cells and tumor cells (Johnstone et al. Blood. 74:1844-1851 (1989); Peters et al., Eur J. Immunol. 19:1469-1475 (1989); Raposo et al., J. Exp Med. 183:1161-1172 (1996); Heijnen et al., Blood 94:3791-3799 (1999); Theiry et al., J. Cell. Biol. 147:599-610 (1999); Wolfers et al., Nature Med. 7:297-303 (2001); van Niel and Heyman, Am J. Physiol Gastrointest Liver Physiol. 283:G251-255 (2002)). Highly purified DC-derived exosomes have been shown to contain certain cytosolic proteins such as tubulin, actin and certain actin-binding proteins as well as MHC class I and II antigens, CD86, ICAM-1, lamp-2, the alphaM-β2 integrin, the tetraspanins CD9 and CD63, and MFGE8/lactadherin (Raposo et al., J. Exp Med. 183:1161-1172 (1996); Theiry et al., J. Cell. Biol. 147:599-610 (1999); Escola et al., J. Biol Chem. 273:20121-20127 (1998); Thery et al., J. Immunol. 166:7309-7318 (2001)).

It has been shown that exosomes derived from DCs pulsed with tumor antigen peptides, wherein the exosomes expose the tumor antigen on their surface, are effective in stimulating an anti-tumor response in mice as efficiently as the DC themselves (Zitvogel et al., Nature Med. 4:594-600 (1998)). Clinical trials using exosomes derived from tumor antigen peptide-pulsed DC have reported initially positive results (Andre et al., Adv Exp Med Biol. 495:349-354 (2001); Morse. et al., Proc. Am. Soc. Oncol. 21 A42, p. 11a (2002)). Exosomes appear to exhibit immunostimulatory capacity and are capable of sensitizing antigen-presenting cells (Zitvogel et al. US20040028692).

Exosomes have also been shown to have some immunosuppressive activity. Certain T cells, as well as melanoma cells, generate exosomes that contain FasL on their surface and are able to stimulate T cell apoptosis, allowing for tumor growth (Andreola et al. J. Exp Med. 195:1303-1316 (2002); Martinez-Lorenzo et al., J. Immunol. 163:1274-1281 (1999)). Additionally, exosomal particles, referred to as tolerosomes, produced by rat intestinal epithelial cells cultured in the presence of INF-γ and digested ovalbumin were able to induce antigen-specific tolerance after injection (Karlsson et al., Eur. J. Immunol. 31:2892-2900 (2001)).

Peche et al. reported that the use of allogeneic donor-derived exosomes prolonged graft survival in rats (Peche et al. Transplantation 76:1503-1510 (2003)). However, the authors also showed a corresponding increase in anti-donor MHC class II alloantibody production, suggesting a concurrent immunostimulatory effect.

Thus, there remains a need to develop a safe, effective method of treating autoimmune diseases and inflammatory disorders. The present invention provides compositions and methods for treating such diseases and disorders.

### 3. SUMMARY OF THE INVENTION

The present invention relates to exosomes having immunosuppressive activity and methods for producing and utilizing said exosomes, Specifically, the exosomes of the invention may be administered to a mammalian host in order to suppress an undesirable immune response.

The exosomes of the invention may be derived from a variety of different cells, including, but not limited to, antigen presenting cells such as dendritic cells and macrophages. The cells from which exosomes are prepared may preferably be genetically engineered and/or treated with an agent, such as but not limited to a cytokine or cytokine inhibitor, prior to exosome harvesting.

In various embodiments, the present invention provides for compositions of exosomes as well as methods for their use as immunosuppressive agents. Diseases and disorders which may be treated according to the invention include, but are not limited to, inflammation and conditions associated with inflammation such as allergy, asthma, arthritis and wound healing, and autoimmune diseases, including but not limited to rheumatoid arthritis and diabetes. Further, in view of the immunosuppressive activity of exosomes, the invention provides for methods of promoting an immune response by antagonizing exosomes, for example to promote anti-tumor immunity in a subject.

### 4. BRIEF DESCRIPTION OF THE FIGURES

FIG. 1A-C. (A) Whole mount transmission electron microscopy (TEM) of exosomes from murine BM-DC. Bar=200 nm. (B) Western blot analysis of exosomes and BMDC lysates for the presence of several exosome-associated proteins. (C) Flow cytometric analysis of murine DC-derived exosomes and DC for expression of MHC I and II, CD11c, CD80 (B7.1), CD86 (B7.2).

FIG. 2. Fluorescence activated cell sorting (“FACS”) characterization of bone marrow dendritic cell (“BMDC”) derived exosomes.

FIG. 3A-D. Trafficking of DC-derived exosomes in vivo. 6 hours post IV injection of PKH67 labeled exosomes, (A) MOMA-1+ and (B) ER-TR9+ macrophages and (C) CD11c+ DC in the spleen were shown to have internalized the exosomes. (D) Internalization of labeled exosomes by subsets of splenic DC assessed by FACS for CD8-alpha and PKH67 at different time points.

FIG. 4A-C. (A) Flow cytometric analysis of mouse bone marrow DC and DC-derived exosomes, where the purified exosomes are from bone marrow DC transduced with Ad.control and Ad.FasL. (B) Western blot of DCs and DC-derived exosomes showing expression of FasL. (C) Transmission electron micrograph of a DC-derived exosome fraction.

FIG. 5. Bar graph demonstrating the suppression of delayed type hypersensitivity (DTH) in mouse footpads treated with DC and exosomes carrying FasL. The “*” denotes significance at p<0.01.

FIG. 6. Bar graph demonstrating DTH suppression in mouse footpads using syngeneic exosomes and DC in comparison to allogeneic exosomes and DC. The “*” denotes significance at p<0.01.

FIG. 7A-B. (A) Bar graph demonstrating the DTH responses in mouse footpads using exosomes and DC infected with Ad.Ψ5 or Ad.FasL injected into wild type and MHC I deficient mice. The “*” denotes significance at p<0.01. (B) Bar graph demonstrating the DTH response in mouse footpads using exosomes and DC infected with Ad.Ψ5 or Ad.FasL injected into wild type or MHC II deficient mice. The “*” denotes significance at p<0.01.

FIG. 8. Bar graph demonstrating the antigen specificity of the immunosuppressive exosomes. The “*” denotes significance at p<0.01.

FIG. 9A-B. (A) Bar graph comparing the DTH-suppressive effect of DC and DC-derived exosomes prepared from either wild type or gld (FasL−/−) mice and infected with either a control adenovirus (psi5) or an adenovirus expressing FasL (FasL), and then injected back into the footpads of wild type mice, previously immunized with KLH, 12 hours prior to injection of KLH into the footpad. (B) Bar graph depicting the DTH suppressive effect of exosomes prepared as in (A) injected back into lpr (Fas−/−) mice, as compared to wild type.

FIG. 10A-B. (A) Graph demonstrating the suppression of disease progression of DC expressing FasL injected into a murine collagen induced arthritis model. The “*” denotes significance at p<0.01. (B) Graph demonstrating the suppression of disease progression of exosomes presenting FasL injected ino a murine collage induced arthritis model. The “*” denotes significance at p<0.01.

FIG. 11A-B. (A) Graph demonstrating suppression of T cell proliferation with the addition of DC expressing vIL-10 in a mixed lymphocyte reaction (MLR). (B) Graph demonstrating suppression of T cell proliferation with the addition of exosomes isolated from DC infected with Ad.vIL-10 in a mixed lymphocyte reaction.

FIG. 12A-B. (A) Bar graph showing the suppression of DTH response of mouse footpads using Ad.vIL-10-transduced DC and exosomes derived from the DC/vIL-10. (B) Bar graph showing the suppression of DTH reaction of mouse footpads using recombinant murine IL-10 treated BMDCs and exosomes derived from DC treated with recombinant murine IL-10. The “*” denotes significance at p<0.01.

FIG. 13A-C. (A) Whole mount transmission electron micrograph of intact or freeze/thawed exosomes from BM-DC transduced with Ad-vIL-10. (B) Western blot of intact or freeze/thawed exosomal preparations from BM-DC transduced with Ad.vlL-10 detecting the presence of Hsc 70. (C) Bar graph demonstrating the inability of membrane-disrupted exosomes isolated from DC infected with Ad.vIL-10 to suppress the DTH reaction.

FIG. 14A-B. (A) Bar graph demonstrating the immunosuppressive effect of MHC II-depleted exosomes isolated from DC infected with Ad.vIL-10. The “*” denotes significance at p<0.01. (B) Bar graph demonstrating the immunosuppressive effect of MHC II-depleted exosomes isolated from DC treated with recombinant IL-10. The “*” denotes significance at p<0.01.

FIG. 15. Graph demonstrating the suppression of disease progression of DC expressing vIL-10 injected into a mouse collagen-induced arthritis model.

FIG. 16A-C. Analysis of the therapeutic effect of exosomes derived from DC/IL-10 in an established collagen-induced arthritis model. (A) Exosomes were isolated from DBA1 mouse bone marrow DC that were infected with either Ad.vIL-10 or pulsed with recombinant mouse IL-10 and administered to mice having established CIA. (B) Exosomes from recombinant IL-10-pulsed DC were divided into two groups and one of them was subjected for three cycles of freeze and thaw to disrupt the membrane prior to administration to mice having established CIA. (C) Exosomes from DC/rmIL-10 were tested in the established CIA mouse compared to direct injection of recombinant mouse IL-10. In A-C, the purified exosomes were injected intravenously at day 32 (as indicated by the arrow) into the DBA1 mice, which were immunized with bovine type II collagen and given LPS at day 28. Mice were monitored periodically by an established macroscopic scoring system expressed as a cumulative value for all paws, with a maximum possible score of 16.

FIG. 17. Increase in footpad swelling in a murine DTH model, where treated footpads were injected with DC/mbmIL-4, exosomes prepared from DC/mbmIL-4, DC/Psi5 (control), exosomes prepared from DC/Psi5 (control), or saline (control).

FIG. 18. Increase in footpad swelling in a murine DTH model, where treated footpads were injected with DC/smIL-4, exosomes prepared from DC/smIL-4, DC/Psi5 (control), exosomes prepared from DC/Psi5 (control), or saline (control).

FIG. 19. Increase in footpad swelling in a murine DTH model, where treated footpads were injected with DC/mbmIL-4, exosomes prepared from DC/mbmIL-4, DC/FasL, exosomes prepared from DC/FasL, DC/Psi5 (control), or exosomes prepared from DC/Psi5 (control).

FIG. 20. Increase in paw swelling in a murine DTH model in either wild type or lpr (Fas−/−) mice, injected with exosomes prepared from DC harvested from either wild type or gld (FasL−/−) mice and modified to express either soluble (smIL-4) or membrane bound (mbmIL-4) IL-4.

FIG. 21A-B. Increase in paw swelling in a murine DTH model after injection with exosomes prepared from either membrane-bound IL-4 enhanced DCs from either (A) syngeneic or (B) allogeneic mice.

FIG. 22. Bar graph showing increase in paw swelling in a murine DTH model, where treated paws (black bars) of either wild type or B7.1 and B7.2 deficient (KO) mice were injected with exosomes prepared from DC treated with either Ad.Psi5 or adenoviral vector containing an IL-4 gene. Untreated paw sizes are represented by open bars.

FIG. 23. Graph depicting the arthritis index of CIA model mice treated with exosomes prepared from DC infected with either Ad.psi=5 (control), Ad.mIL-4, or Ad.mbmIL-4.

FIG. 24A-B. DTH reactions in(A) mice primarily treated with DC/IL-4-derived exosomes or (B) mice administered CD11C from primary subjects.

FIG. 25. Graph showing occurrence of hyperglycemia in mice treated with either saline, exosomes from untreated DC, or exosomes prepared from DC/IL-4.

FIG. 26. Bar graph showing the footpad swelling 48 hours after the administration of untreated whole serum and whole serum treated with beads to mice in the DTH model.

FIG. 27. Bar graph showing DTH suppression in mouse footpads using serum, microvesicles, and exosomes from a KLH immunized mouse.

FIG. 28. Bar graph showing DTH suppression in mouse footpads using various serum fractions where swelling was measured 48 hours after boost.

FIG. 29. Bar graph showing DTH suppression in mouse footpad using various serum fractions, where serums are isolated from KLH and OVA immunized mice where swelling was measured 48 hours after boost.

FIG. 30. Electron micrograph of exosomes isolated from mouse serum.

FIG. 31. FACS analysis of serum-derived exosomes labeled with beads carrying anti-MHC class II.

FIG. 32. Bar graph showing increases in paw swelling in a murine DTH model (where the DTH is against KLH), where the treated paws were injected with either (a) exosomes collected from serum of a KLH-immunized mouse (Group I), (b) exosomes collected from serum from a naive mouse (Group II), or (c) saline (Group III). The treated paws are represented by black bars, the contralateral paws by open bars.

FIG. 33. Bar graph showing increases in paw swelling in a murine DTH model (where the DTH is against KLH), where the treated paws were injected with either (a) exosomes collected from serum of a KLH-immunized mouse; (b) exosomes collected from serum of a KLH-immunized mouse and preabsorbed with anti-MHCII antibody; (c) exosomes collected from serum of a KLH-immunized mouse that are MHC class II positive; (d) an exosome depletion control preadsorbed with anti-IgG antibody; or (e) saline.

FIG. 34. Bar graph showing increases in paw swelling in a murine DTH model, wherein (a) serum-derived exosomes from FasL deficient gld (FasL−/−) mice were administered to a wild type recipient; (b) serum-derived exosomes from wild type mice were administered to a wild-type recipient; (c) serum-derived exosomes from gld mice were administered to a lpr (Fas−/−) recipient, (d) serum-derived exosomes from wild type mice were administered to a lpr recipient, or (e) saline was administered as a control.

FIG. 35. Bar graph showing DTH increases in paw swelling in a murine DTH model, treated with serum derived exosomes collected 14 days after immunization of the exosome donor animal, administered 14 days following immunization of the recipient animal.

FIG. 36. Graph showing the pain in VAS measured at six times after the injection for of three groups: the Orthokine® serum and two groups of Triam.

FIG. 37. Graph showing the pain in SESaff (Affective Pain Scale) measured at six times after the injection for of three groups: the Orthokine® serum and two groups of Triam.

FIG. 38. Graph showing the pain in SESsens (Sensitive Pain Scale) measured at six times after the injection for of three groups: the Orthokine® serum and two groups of Triam.

FIG. 39. Graph showing the pain Oswestry Score measured at four times after the injection for of three groups: the Orthokine® serum and two groups of Triam.

FIG. 40A-F. Transmission electron micrographs (TEM) of exosome-enriched fractions from the Orthokine® serum ((B) is an image generated from filtered serum).

### 5. DETAILED DESCRIPTION OF THE INVENTION

For clarity and not by way of limitation, the detailed description of the invention is divided into the following subsections:

(i) cell sources for exosomes;

(ii) conditioning cells for exosome harvest;

(iii) exosome preparation from cells;

(iv) exosome preparation from serum;

(v) exosome-containing compositions;

(vi) methods of immunosuppression; and

(vii) methods of antagonizing exosome-mediated immunosuppression.

### 5.1 Cell Sources for Exosomes

The exosomes of the present invention may be derived from a variety of different cells, including, but not limited to, antigen presenting cells (“APCs”) such as dendritic cells (“DCs”) and macrophages, which may be harvested, for example, from tissues such as the bone marrow, spleen, lymph node, or thymus, or from the peripheral blood or serum derived therefrom. The scope of the invention further encompasses specialized antigen presenting cells, such as Langerhans cells of the skin or Kuppfer cells of the liver, which may be prepared from their tissue of origin. Methods of harvesting APCs and DCs in particular are known in the art.

As demonstrated in working examples set forth below, the immunosuppressive activity of exosomes has been observed to be MHC class II antigen dependent, and to be much higher when the relationship between exosome donor and recipient is syngeneic, rather than allogeneic. Accordingly, it is desirable to maximize the relatedness of donor and recipient. Therefore, while the invention encompasses the use of exosomes from one mammalian species for immunosuppression in another species, preferably the species of donor and intended recipient are the same and/or preferably the MHC class II antigens of donor and intended recipient are the same (or are substantially similar or compatible, using the considerations employed for contemplated tissue transplants, for example), and/or preferably the donor and intended recipient are the same (autologous) or are familialy related (brother/sister; sister/sister; parent/child). Similarly, because the immunosuppressive activity of exosomes is antigen specific, in specific non-limiting embodiments of the invention the exosome donor may be immunized with the antigen, a reaction to which is to be suppressed in the recipient.

### 5.2 Conditioning Cells for Exosome Harvest

In preferred non-limiting embodiments of the invention, APCS are conditioned to enhance the immunosuppressive activity of exosomes prepared therefrom. “Conditioned,” as that term is used herein, includes (i) exposure of APCs, in vitro or in vivo, to an enhancing agent, as well as (ii) genetically engineering the APCs to express an enhancing agent.

Enhancing agents may be cytokines, cytokine antagonists, and NFκB antagonists, and include, but are not limited to, TGF-β, IL-10, CTLA4-Ig, sCD40-Ig, IL-4, IL-13, FasL, IL-1 receptor antagonist protein (“IRAP”), vIL-10, sICAM-1, sICAM-3, and TRAIL. In preferred, non-limiting embodiments, the enhancing agent is IRAP or IL-10 or IL-4 or a combination thereof. In a specific, non-limiting embodiment, where the enhancing agent is administered to APCs, for example in cell culture, the concentration of IRAP may be about 5 μg/ml, or the concentration of IL-10 may be about 1000 U/ml, or the concentration of IL-4 may be about 1000 U/ml. Optionally, where a specific antigen or a specific antigen source is known, such specific antigen or specific antigen source (e.g., fixed or attenuated infectious agent) may be added to the culture as an enahncing agent in a non-toxic, non-pathogenic amount.

In one set of embodiments of the invention, APCs may be genetically engineered to express a heterologous “enhancing gene” which encodes an enhancing agent. Such “enhancing genes” include, but are not limited to, nucleic acids encoding TGF-β, IL-10, CTLA4-Ig, sCD40-Ig, IL-4, IL-13, FasL, IRAP, VIL-10, sICAM-1, sICAM-3, and TRAIL, operably linked to a promoter element that is active in the APCs. In preferred embodiments, the enhancing gene is FasL, IL-10, IL-4 or IRAP. The enhancing gene product may be expressed on the surface of the exosomes (for example, membrane bound) or in the interior of the exosome. Alternatively, APCs may be engineered to express an enhancing gene encoding an angiogenic factor, such as but not limited to Dell, or a sorting and localization signal. The enhancing gene may be introduced using methods known in the art, including transfection, transduction, electroporation, microinjection, etc. The enhancing gene may optionally be incorporated into a suitable expression vector to facilitate its introduction. In non-limiting embodiments of the invention, the expression vector may be a viral vector. The viral vector may be, for example, a retrovirus, adenovirus, adeno-associated virus (AAV), or herpes simplex virus (HSV) vector. In a specific preferred embodiment of the invention, the viral vector is derived from adenovirus. (See generally, Horwitz, M. S., “Adenoviridae and Their Replication,” in Virology, 2nd edition, Fields et al., eds., Raven Press, New York, 1990). Recombinant adenoviruses have advantages for use as expression systems for nucleic acid molecules coding for proteins that are foreign to the adenovirus carrier, including tropism for both dividing and non-dividing cells, minimal pathogenic potential, ability to replicate to high titer for preparation of vector stocks, and the potential to carry large inserts. See Berkner, K. L., 1992, Curr. Top. Micro Immunol, 158:39-66; Jolly D., 1994, Cancer Gene Therapy, 1:51-64.

In specific non-limiting embodiments of the invention, the enhancing gene may be contained in an adenoviral vector derived from adenovirus serotype 2 (Ad 2) or serotype 5 (AD5) which has a substantially deleted E1 and E3 region. Other adenovirus serotypes can also be used as backbones for the adenoviral vector including, inter alia, Ad 6, Ad 9, Ad 12, Ad 15, Ad 17, Ad 19, Ad 20, Ad 22, Ad 26, Ad 27, Ad 28, Ad 30 and Ad 39. From these enumerated adenovirus serotypes, Ad 2 and Ad 5 are preferred.

A nucleic acid comprising an enhancing gene operably linked to suitable promoter element, optionally contained in a viral vector, may, for example, be provided to an APC via a delivery system, such as encapsulation in a liposome, microparticle, or microcapsule.

### 5.3 Exosome Preparation from Cells

Exosome preparation from cells is known in the art; see, for example, Raposo et al., J. Exp. Med. 183:1161 (1996).

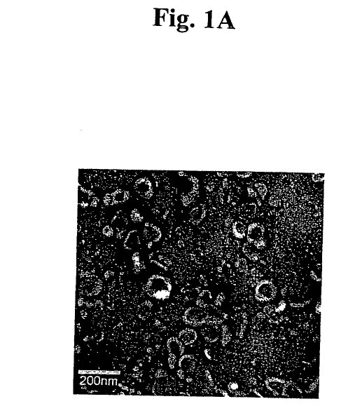

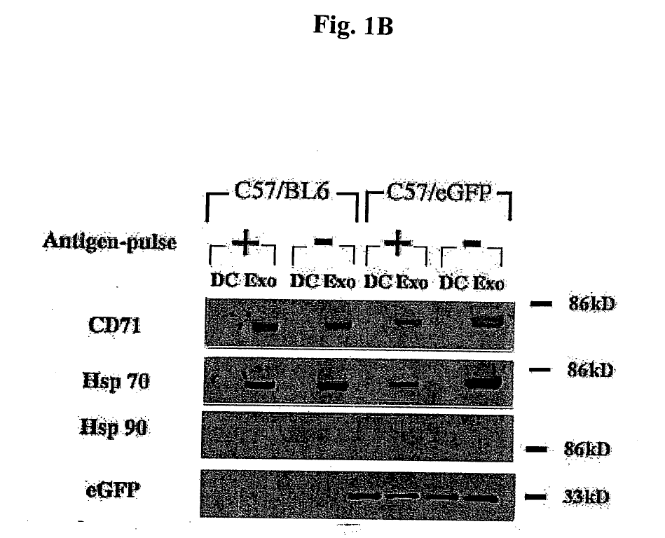

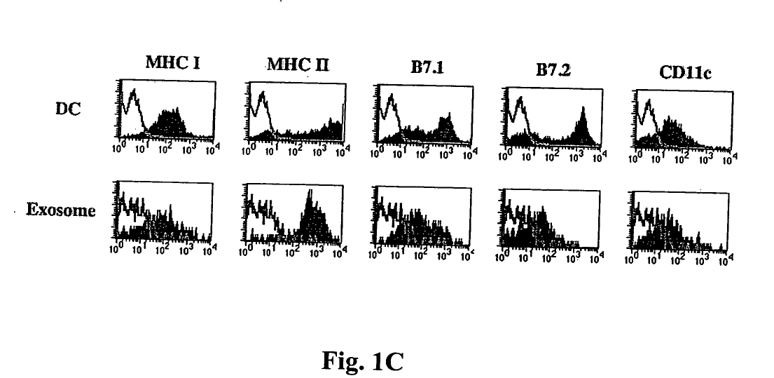

In specific non-limiting embodiments of the invention, exosomes may be prepared from a culture of APCs, preferably conditioned by an enhancing agent (either in the culture medium or through genetic engineering), as follows. Culture supernatant may be collected and subjected to three successive centrifugations at 300 g for 5 minutes, 1,200 g for 20 minutes, and 10,000 g for 30 minutes, to eliminate cells and debris, followed by centrifugation for one hour at 100,000 g. To remove excess serum proteins, the exosome pellet may then be washed with PBS and the centrifuged again at 100,000 g for 1 hour, after which the resultant pellet may be resuspended in PBS. Exosomes may be quantified by a micro Bradford protein assay (Bio-Rad, CA), and preferably an amount of exosomes corresponding to 1 mg protein, as determined by the assay, may be suspended in 20 ml PBS. The integrity of the exosomes may optionally be confirmed by electron microscopy (see FIG. 1A), and the exosomes may optionally be characterized by FACS analysis for characteristic surface markers (see Section 6s 6 and 7, below).

### 5.4 Exosome Preparation from Serum

Exosomes which may be used for immunosuppression according to the invention may be collected from the serum of a suitable subject. Preferably, the subject is also the intended recipient of the serum-derived exosomes (autologous administration). If the exosome donor and recipient are not the same, due to the antigen specificity and MHC class II dependence of exosome-mediated immunosuppressive activity, it is preferable that the donor and recipient are MHC class II antigen compatible and/or that the donor has had exposure to the antigen to which immunity is desirably suppressed in the recipient.

As exosomes are small particles, 30-100 nM in size, they may be recovered in serum by removing the larger cellular elements from a peripheral blood sample, for example, but not by way of limitation, by centrifugation at 1500 g for 10 minutes.

Preferably, the exosomes are more rigorously purified by sequential centriftigation steps. For example, but not by way of limitation, the method outlined above for preparing exosomes from a cell culture supernatant, or an equivalent method, may be used. Such a method preferably involves use of a laboratory ultracentrifuge. In one non-limiting example, exosomes may be separated from serum (collected from peripheral blood using standard laboratory techniques), by three successive centrifugations at 1200 g for 5 minutes, 1,200 g for 20 minutes, and 10,000 g for 30 minutes, followed by centrifugation for one hour at 100,000 g, washing the resulting pellet with PBS, resuspending in PBS and then centrifuging again at 100,000 g for 1 hour, after which the resultant pellet may be resuspended in PBS.

In preferred embodiments of the invention, peripheral blood may be incubated in the presence of beads to stimulate the production of cytokines prior to serum and exosome collection. Beads which may be used for this purpose include, but are not limited to, glass or plastic beads between 0.5 and 10 mm or between 0.5 and 5 mm in diameter, optionally treated with an agent, such as CrSO₄, which stimulates lymphocyte proliferation (Mignini et al., 2004, Preventive Med 39(4) 767-775; Rhee et al., 2002, Clin Exp Immunol 127(3):463-469). In one preferred, non-limiting embodiment of the invention, glass beads, 2.5 mm in diameter, having a surface area of 21 mm,² of medical grade, surface modified by incubation in 50% CrSO₄ (Merck, Germany) for 5 minutes, then washed with distilled water until the pH was the same as that of the distilled water and the conductivity of the wash solution was less then 0.3 μS, may be used. The treated beads may be placed in a suitable container, such as a microtiter plate, centrifuge tube, culture tube, or syringe, and then sterilized (e.g. by autoclaving or gamma irradiation). Peripheral blood may then be introduced into the bead-containing container, and then incubated, aseptically, at 37° C., 5% CO₂, for example for 24 hours. Serum may then be collected from the bead/blood suspension by centrifugation, for example at 3500 rpm for 10 minutes. Typically, 20 percent of the total original peripheral blood volume may be recovered. The resulting serum containing exosomes may then be stored at −20° C. Orthokine® serum is prepared in this way (see U.S. Pat. Nos. 6,759,188 and 6,713,246).

In a related, specific, non-limiting embodiment of the invention, IRAP may be added to the peripheral blood sample prior to, or as an alternative to, incubation with beads. For example, 5 μg IRAP per ml of peripheral blood may be added.

In a preferred, non-limiting embodiment of the invention, a concentrated preparation of exosomes may be prepared by collecting serum from peripheral blood, optionally incubated with beads and/or IRAP, by centrifugation to remove the formed blood elements (e.g., at 3000-5000 g for 10 minutes), followed by ultracentrifugation, for example, at 100,000 g, for 1 hours. The resulting pellet may be resuspended in physiologic saline, and then preferably sterilized (e.g., by filtration through a 0.2 μm filter). The volume into which the pellet is suspended determines the concentration of the exosomes. Preferably, the concentration is between about 100 ml serum: 1 ml exosome concentrate (“100× concentrate”) and 2 ml serum: 1 ml exosome concentrate (“2× concentrate”), preferably between about 50 ml serum: 1 ml exosome concentrate (“50× concentrate”) and 5 ml serum: 1 ml exosome concentrate (“5× concentrate“), and preferably about 10 ml serum: 1 ml exosome concentrate (“10× concentrate”).

### 5.5 Exosome-Containing Compositions

The present invention provides for exosome-containing compositions, wherein exosomes are suspended in a suitable pharmaceutical carrier.

The compositions of the invention may be characterized by a concentration of exosomes that is concentrated relative to their concentration in serum in either the average subject or the intended recipient of the exosomes. In non-limiting embodiments, the concentration, relative to serum, may be between about 100×-2×, or between 50× and 5×, or is about 10×.

The compositions of the invention may comprise exosomes that are obtained from APCs or peripheral blood treated with an enhancing agent, as set forth in the foregoing sections.

The compositions of the invention may comprise exosomes that are prepared by ultracentrifugation.

In one preferred, non-limiting set of embodiments, the present invention provides for a pharmaceutical composition comprising exosomes, prepared by culturing APCs of a subject conditioned by an enhancing agent, and then isolating exosomes from the culture medium of said conditioned APCs.

In another preferred, non-limiting set of embodiments, the present invention provides for a pharmaceutical composition comprising exosomes prepared by incubating peripheral blood with glass beads, collecting serum from the peripheral blood, and isolating exosomes from the serum by ultracentrifugation.

In another preferred, non-limiting set of embodiments, the present invention provides for a pharmaceutical composition comprising exosomes prepared by incubating peripheral blood with glass beads in the presence of an enhancing agent, preferably IRAP, collecting serum from the peripheral blood, and isolating exosomes from the serum, preferably by ultracentrifugation, and preferably to produce a concentrated exosome preparation.

### 5.6 Methods of Immunosuppression

The present invention provides for methods for decreasing, inhibiting, or preventing an immune response in a subject in need of such treatment, comprising administering, to the subject, an effective amount of APC-derived exosomes. The decrease/inhibition/prevention may be evidenced by a decrease in inflammatory parameters, such as clinical signs of inflammation (swelling, redness, warmth, pain, limited joint mobility, rash, inflammatory neuropathy, meningitis, encephalitis), clinical signs of allergy or asthma (sneezing, itching, coughing, rash, hives, wheezing), clinical signs of inflammatory bowel disease (cramps, blood and/or mucus in stools) or clinical markers, such as CRP, ESR, WBC.

Diseases and disorders in which decreasing, inhibiting, or preventing an immune response may be desirable include, but are not limited to, arthritis, allergy, asthma, or an autoimmune disease such as, but not limited to, rheumatoid arthritis, juvenile rheumatoid arthritis, systemic lupus erythematosis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo. Other conditions which may desirably be treated according to the invention include diseases such as muscular dystrophy, and conditions in which inflammation can interfere with proper healing, such as an accidental or iatrogenic wound in soft tissue, ligament, or bone, or tissue damaged by a non-immune event, for example, heart muscle following myocardial infarction.

The methods of the invention involve administering, to a subject in need of such treatment, an effective amount of exosomes, prepared according to the invention. For example, exosome compositions as described in the foregoing section may be administered. Exosomes may be administered by any clinical route, but preferably are administered intravenously, intramuscularly, intraarticularly, subcutaneously, intrathecally,or by local injection or instillation, for example during a surgical procedure.

The amount of exosomes to be administered may be as follows, or may be as determined on a clinical basis, for example, on a subject-by-subject basis. In a specific, non-limiting embodiment, an amount of exosomes having between about 5 and 100 μg protein, or an amount of exosomes having about 50 μg protein, may be administered per one kilogram of subject. The phrase “exosomes having a particular amount of protein” means that the amount of protein present in an exosome preparation is quantified (for example, by the Bradford protein assay as set forth in section 5.3, or another standard technique for measuring protein), and the amount of protein is used as an index of the dose of exosome adminsitered. In one set of specific non-limiting embodiments of the invention, a human subject may be administered an amount of exosomes derived from serum collected from between about 20-50 ml, between about 50-100 ml, between about 100-200 ml, between about 200-300 ml, between about 300-400 ml, or between about 400-500 ml, of peripheral blood. In another set of specific non-limiting embodiments of the invention, a human subject may be administered an amount of exosomes having between about 100 μg and 5 mg protein, or between about 500 μg and 2 mg protein.

In particular non-limiting embodiments, the present invention provides for a method of inhibiting an immune response in a subject in need of such treatment, comprising administering, to the subject, an effective amount of exosomes prepared from a culture of antigen presenting cells. As used herein, a “culture of antigen presenting cells” is a culture of collected cells enriched for antigen presenting cells by methods known in the art; such a culture is not necessarily 100 percent pure.

### 5.7 Methods of Antagonizing Exosome-Mediated Immunosuppression

Because APC-derived exosomes have been found to be immunosuppressive, they may, under certain conditions, exert a negative effect, for example, in the context of suppressing the immune response of a host to a tumor or to an infection. Accordingly, the present invention provides for a method of inhibiting such undesirable immunosuppression comprising administrating, to a site where increased immunity is desired, an effective amount of an APC-derived exosome inhibitor. Such an inhibitor may, for example, be an antibody directed at an exosome-associated antigen, such as, for example, transferrin or any of the surface molecules depicted in FIGS. 1C, 2 and 4A.

### 6. EXAMPLE

### Characterization of Exosomes-I

To demonstrate that the immuno-regulatory role of DC-derived exosomes, DCs were generated from C57BL/6 mouse bone marrow precursors cultured at high density in GMCSF/IL-4. Exosomes produced by the DCs were then isolated from the culture media by differential centrifugation and characterized by electron microscopy, Western blot and flow cytometry.

### 6.1 Materials and Methods

Mice: Female C57BL/6 (H-2Kb) mice and male DBA1/LacJ (H-2Kq) mice, all 7-8 wk of age, were purchased from the Jackson Laboratory (Bar Harbor, Me.). Animals were maintained in a pathogen-free animal facility at the University of Pittsburgh Biotechnology Center (Pittsburgh, Pa.).

Generation and culture of bone marrow-derived DC: Bone marrow-derived DC (BMDC) were generated as described by Kim et al., J. Immunol. 166:3499-3505 (2001). Briefly, bone marrow was harvested from mouse tibias and femurs and passed through a nylon mesh to eliminate small pieces of bone and debris. Contaminating erythrocytes were lysed with 0.83 M NH4Cl buffer, and lymphocytes were depleted with a cocktail of Abs (RA3-3A1/6.1, anti-B220; 2.43, anti-Lyt2; GK1.5, anti-L3T4; all from American Type Culture Collection, Manassas, Va.) and rabbit complement (Accurate Chemical and Scientific, Westbury, N.Y.) on day 0. The cells were then cultured for 24 h in complete medium (CM; RPMI 1640 containing 10% FBS, 50 μM 2-ME, 2 mM glutamine, 0.1 mM nonessential amino acids, 100 μg/ml streptomycin, and 100 IU/ml penicillin) to remove the adherent macrophages. The nonadherent cells were then placed in fresh CM containing recombinant murine GM-CSF (1000 U/ml) and recombinant murine IL-4 (1000 U/ml) on day 1. Cells were cultured for 4 days and harvested for adenoviral transduction or recombinant cytokine treatment on day 5.

For adenoviral infection, 1×10⁶ DC/well were plated on 24-well plates, and 5×10⁷ PFU of each recombinant adenovirus was added in a total volume of 1 ml serum-free medium. After incubation for 24 h at 37° C., the cells were collected and washed 5 times in PBS, and fresh media added. On day 7, infected DC and exosomes were recovered, extensively washed and injected into animals.

Exosome Purification: Exosomes were prepared from the cell culture supernatant of day 7 BMDC cultures by differential centrifugation as previously described (Raposo et al., J. Exp Med. 183:1161-1172 (1996)). Briefly, recovered culture supernatant from each BMDC culture was subjected to three successive centrifugations at 300 g (5 min), 1,200 g (20 min), and 10,000 g (30 min) to eliminate cells and debris, followed by centrifugation for 1 h at 100,000 g. To remove excess serum proteins, the exosome pellet was washed with a large volume of PBS and centrifuged at 100,000 g for 1 h, and finally resuspended in 120 μl of PBS for further studies. The exosomes were quantified by a micro Bradford protein assay (Bio-Rad, CA). Each batch was standardized by protein content and 1 μg was suspended in 20 μl of PBS for in vivo mouse studies. For MHC II adsorption, 100 μl of washed anti-mouse MHC II paramagnetic beads (Miltenyi Biotech) were incubated with pre-diluted exosomes (1 μg/20 μl) for 1 hour, 4° C. with gentle shaking. After magnetic separation, the fraction not retained in the micro-centrifuge tube was adjusted to the original volume with PBS. The freeze/thaw of pre-diluted exosomes was performed by 3 separate rounds of snap freezing in a dry ice/ethanol bath and subsequent warming in 37° C. bath. Contaminating concentrations of IL-10 and vIL-10 in final exosome preparations were determined by IL-10 ELISA (Endogen).

Electron Microscopy: Exosomes were purified by differential centrifugation, 10 ml loaded on a Formvar/carbon coated grid, negatively stained with 10 μl neutral 1% aqueous phosphotungastic acid and viewed using a JEOL-1210 computer controlled high contrast 120 kv transmission electron microscope.

Protein Analysis: For cells, the cytosol was separated from the total membranes by homogenization in 10 mM triethanolamine, 1 mM EDTA, 10 mM acetic acid, 250 mM sucrose, pH 7.4, supplemented with CLAP (chymotrypsin, leupeptin, aprotinin, and pepstatin, 100 μM each), by 60 passages through a 25-G needle. The supernatant was cleared from nuclei and cell debris by centrifugation at 1,200 g. Total membranes were recovered in the pellet after centrifugation for 1 h at 100,000 g. 10 μg of cell lysate or exosomal preparation were then separated on 5-20% gradient SDS-PAGE, transferred onto nitrocellulose and detected by western blot using an enhanced chemiluminescence detection kit (Amersham).

FACS Analysis: DCs were defined by phenotypic analysis for expression of CD11b, CD11c, CD80, CD86, and MHC class I and class II in the majority of the cultured cells (60-95%) by FACScan (Becton Dickinson, Sunnyvale, Calif.). For exosomes, 30 μg of pelleted exosomes were incubated with 10 μl of 4 μm diameter aldehyde/sulfate latex beads (Interfacial Dynamics, Portland, Oreg.) for 15 minutes at room temperature in a 30-100 μl final volume, followed by 2 hr. with gentle agitation in 1 ml PBS. The reaction was stopped by 30 min. incubation in 100 mM glycine. Exosome coated beads were washed three times in FACS wash buffer (3% FCS, and 0.1% NaN3 in PBS) and resuspended in 500 μl FACS wash. Beads were incubated for 1 hour with each primary antibody, followed when necessary by incubation in FITC-conjugated secondary antibody, washed and analyzed on a FACSCaliber (Becton Dickinson, San Diego, Calif.). Data acquisition and analysis was performed using Lysis II FACScan software (Becton Dickinson).

### 6.2 Results

Ultrastructural analysis of exosome pellets by whole mount transmission electron microscopy showed a significant enrichment of the characteristic saucer shaped exosomes, 40-90 nm in diameter (FIG. 1A). Western blot analysis demonstrated that the DC-derived exosomes were positive for the exosome associated proteins CD71 and Hsp70 (FIG. 1B), but negative for proteins not found in exosomes, such as Hsp90, the invariant chain, and calnexin. To further demonstrate the intact vesicular nature of the exosome fraction, exosomes were purified from BMDC of eGFP/C57 mice, since the majority of cells within the animal constitutively express the marker protein eGFP (FIG. 1B). Western analysis of the highly enriched exosome fraction showed a significant level of full-length eGFP, suggesting that the soluble eGFP is encapsulated within the protective environment of the lumen of BMDC derived exosomes.

The DC-derived exosome fraction was further examined by flow cytometry for surface proteins. Exosomes were recovered after the 100,000×g spin, bound to latex beads and stained with several monoclonal antibodies against murine DC-associated leukocytic marker proteins. The surface of exosomes stained positive for high levels of MHC II, with more moderate levels of MHC I, CD11C, CD80 (B7.1) and CD86 (B7.2) detected (FIG. 1C). Taken together, these data demonstrate the ability to enrich for intact exosomes that contain many of the markers of DC-derived exosome associated proteins as described by others (Stoorvogel et al., Traffic. 3:321-330 (2002); Raposo et al., J. Exp Med. 183:1161-1172 (1996); Kleijmeer et al., Traffic. 2:124-137 (2001)).

### 7. EXAMPLE

### Characterization of Exosomes—II

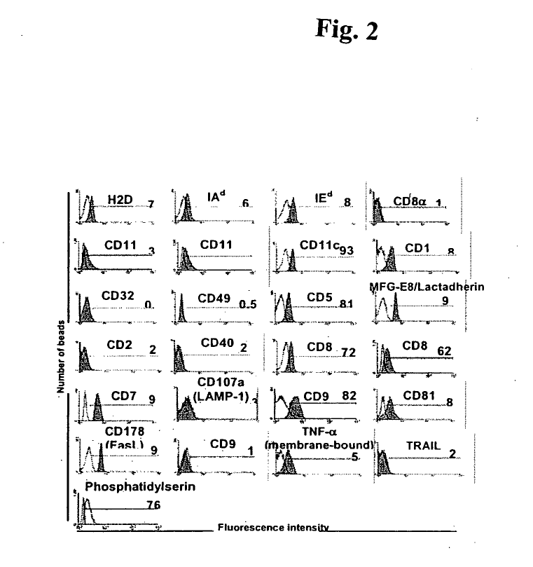

Surface antigens. FIG. 2 shows the results of FACS analysis demonstrating the surface phenotype of DC-derived exosomes. The exosomes were incubated with 4.5 μm beads coated with CD11b (for analysis of MHC Class II) or MHC Class II (IA^{d}) antibodies. Beads were used to increase the size of the exosomes so that they would be detectable by FACS. The bead-coated exosomes were then labeled with PE mAbs for the indicated proteins. In addition to the surface antigens indicated in FIG. 2, the bone marrow-derived exosomes were also positive for CD11c, CD14, CD54, MFG-E8, CD80, CD86 and CD9. A significant percentage of the exosomes were positive for CD11a, CD11b and membrane-bound TNF-alpha, but were negative for CD8-alpha, CD32, CD49d, CD25, CD40, CD107a (Lamp-1), CD95 and Trail. Interestingly, 99 percent of the exosomes were positive for FasL (CD178) even though only a small percentage of the DC from which they were prepared were FasL positive. This result suggests that FasL is preferentially sorted into exosomes where, without being bound to any theory, it appears to play an essential role in conferring the observed therapeutic effects.

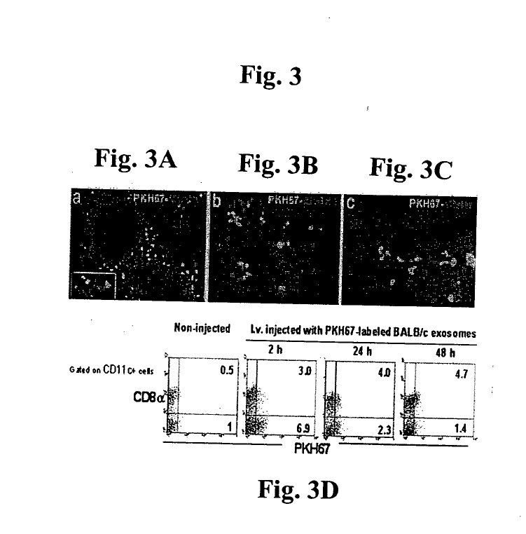

Trafficking. FIG. 3A-D shows trafficking of DC-derived exosomes in vivo. BDMC-derived exosomes were labeled with PKH67, injected IV into mice, and the mice analyzed starting at 2 hours post-injection (Kim et al., J. Immunol. 166:3499-3505 (2001)). The labeled exosomes were detected in MOMA-1+ macrophages, ER-TRP+ macrophages and in CD11c+ DC in the splenic marginal zone. Within the CD11c+ DC, the exosomes were found to be associated with Lamp-1+ endocytic vesicles. Moreover, initially the exosomes were taken up by CD8-alpha negative CD11c+ cells with the percent of CD8-alpha+ Dc positive for the labeled exosomes increasing over time. After 24 hours, the exosomes were shown to be associated with CD11c+ DC in T cell areas. Also, analysis of the exosome positive DC showed that they did not upregulate DC maturation markers (IA^{b}, CD86 or CD54). These results suggest that DC-derived exosomes are efficiently internalized by immature DC found in the spleen as well as by macrophages without inducing DC maturation, but also without affecting the ability of the DC to mature. The working model for exosome function is thus that they interact with a subset of antigen presenting cells in the spleen and lymph node, which in turn suppress T cell responses, resulting possibly in induction of a regulatory T cell population.

### 8. EXAMPLE

### Exosomes Derived from DC/FasL Have Immunosuppressive Activity

The notation “DC/′X″”, where ‘X’ is an agent such as FasL, IL-10, IL-4, etc., indicates that dendritic cells were either engineered to express X or that DC were exposed to X prior to the preparation of exosomes from the dendritic cells.

### 8.1 Exosomes Derived from DC/FasL Carry Exongenous FasL

The following example demonstrates that exosomes isolated from DC transduced with an exogenous gene is capable of presenting the gene product. In particular, the DC-derived exosomes exhibit FasL when the DC has been transduced with adenoviral vector carrying the FasL gene.

### 8.1.1 Materials and Methods

DC generation: Bone marrow derived DC was generated as described previously (Whalen et al., Mol Ther. 4:543-550 (2001); Kim et al., J. Immunol. 166:3499-3505 (2001); Kim et al., Mol Ther. 6:584-590 (2002)). Briefly, bone marrow was collected from tibias and femurs of C57BL/6 mice. Contaminating erythrocytes were lysed and lymphocytes depleted with a cocktail of antibodies (RA3-3A1/6.1, anti-B220; 2.43, anti-Lyt2; GK1.5, anti-L3T4; all from ATCC, MD). The cells then were cultured for 24 hr in complete media (CM) to remove the adherent macrophages. The non-adherent cells were then placed in fresh CM containing 1000 U/mls of mGM/CSF and mIL-4. Cells were cultured for 4 days and harvested for adenoviral transduction. For adenoviral infection, 1×10⁶ DC were mixed with 5×10⁷ PFU viruses in total volume of 1 ml serum-free media. After incubation for 24 hr, DC were washed intensively three times with PBS and incubated for a further 48 hr. On day 8, culture supernatant was collected for exosomes purification and recovery of the infected DC.

Exosomes isolation: Exosomes were isolated as previously described with minor modification (Raposo et al., J. Exp Med. 183:1161-1172 (1996)). Collected culture supernatants were centrifuged at 300 g for 10 min, 1200 g for 20 min, and 3000 g for 30 min. The supernatant from the final centrifugation was centrifuged again at 100,000 g for 1 h in the ultra-centrifuge. The exosome pellet was washed in saline, centrifuged at 100,000 g for 1 h, and resuspended in saline.

Flow cytometry: For phenotypic analysis of DCs, the DCs were stained with PE- or FITC-conjugated monoclonal antibodies against murine surface molecules (CD11b, CD11c, CD80, CD86, H-2Kb, I-Ab, and appropriate isotype controls).

For FACS analysis, the exosomes were incubated with 5 μl of 4 μm diameter aldehyde/sulfate latex beads in a final volume of 20 μl for 15 min at room temperature. After addition of 10 mg bovine serum albumin (BSA) into the each beads/exosomes sample, the incubation was continued for 15 min. One ml of saline was added in, followed by 75 minutes of incubation with gentle shaking. Reaction was stopped by incubation for 30 min with 100 mM glycine. Exosomes-coated beads were stained with antibodies, washed twice in FACS buffer (3% fetal bovine serum (FBS) and 0.1% NaN3 in saline) and resuspended in 400 μl FACS buffer. DC and exosomes were examined by FACScan (Becton Dickenson, CA).

Electron Microscopy: Exosomes were purified by differential centrifugation, 10 ml loaded on a Formvar/carbon coated grid, negatively stained with 10 ml neutral 1% aqueous phosphotungastic acid and viewed using a JEOL-1210 computer controlled high contrast 120 kv transmission electron microscope. Bone marrow derived DCs were isolated. Half of the isolated DCs were infected with Ad.FasL at day 5. Exosomes were isolated as described above.

Protein analysis and Western Blotting: Five micrograrns of exosome proteins and DC lysates were separated by 12% sodium dodecyl sulfate (SDS) polyacrylarnide gel electrophoresis (PAGE). The proteins were blotted onto a nitrocellulose membrane (Amersham). After blocking, the membrane was revealed with antibodies followed by horseradish peroxidase and detected by enhanced chemiluminescence (Perkin Elmer Life Science) using X-ray films.

### 8.1.2 Results

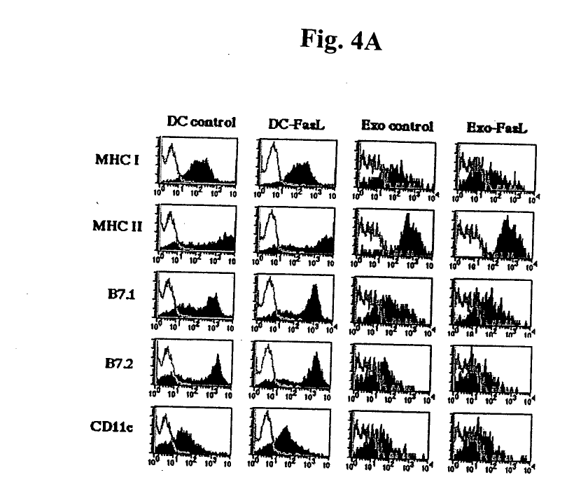

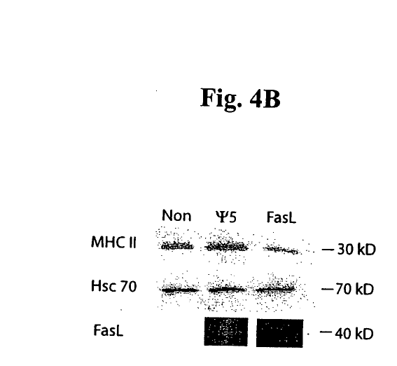

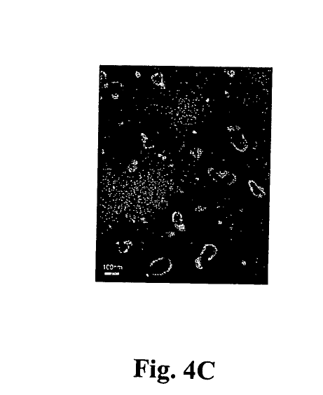

After extensive washing one day post-transduction with Ad.eGFP or Ad.FasL, the infected murine DC were cultured for 48 hours and exosomes isolated from the supernatant by differential centrifugation. 10⁶ murine bone-marrow DCs produced 1-2 μg of exosomes over the 48 hour period. The DCs and the isolated DC-derived exosomes were then analyzed for the presence of MHC and co-stimulatory molecules by incubation with antibodies coupled to latex-beads followed by flow cytometry. As shown in FIG. 4A-C, exosomes derived from the Ad.eGFP and Ad.FasL transduced DCs as well as the control transduced DC were positive for MHC class I and II molecules, CD11c and the co-stimulatory molecules CD80 and CD86 (FIG. 4A). In addition, both DC/FasL and DC/FasL-derived exosomes were shown to be positive for the ˜40 Kda human FasL transgene by Western analysis using an anti-FasL antibody (FIG. 4B). These results demonstrate that exosomes derived from DC transduced with Ad/FasL contained non-proteolyzed, intact FasL as well as MHC, co-stimulatory molecules, and CD11c. Analysis of exosome fraction by EM showed a significant number of saucer-shaped vesicles, characteristic of exosomes (FIG. 4C).

### 8.2 Local Administration of Exosomes Presenting FasL Reduced Delayed Type Hypersensitivity (“DTH”) Associated Swelling Both in Treated and Contralateral Paws in a Footpad Swelling Assay

To test whether DC genetically modified to express FasL, as well as exosomes derived from the modified DC, are able to suppress inflammation in vivo, a DTH mouse model was utilized. In this model, mice are immunized to a specific antigen, keyhole limpet hemocyanin (KLH) or ovalbumin (OVA), and then a Th1-mediated inflammatory response induced 10-14 days post-immunization by injection of the specific antigen into the hind footpads.

### 8.2.1 Materials and Methods

After isolation from bone marrow, DCs were infected with Ad.Ψ5 or Ad.FasL. Exosomes were purified as described in Section 8.1.

Exosomes administration into a DTH model: C57BL/6 mice were sensitized by injecting 100 μg antigen (KLH or OVA) emulsified 1:1 in Freund's complete adjuvant (FCA) at a single dorsal site. Ten days later, one hind footpad of the immunized mouse was injected with a million of DC or 1 μg of exosomes derived from DC, 12 h before challenge with antigen. The contra-lateral footpad received an equal volume of saline instead of DC or exosomes. Mice were challenged in both footpads by injecting 20 μg antigen dissolved in 20 μl saline. Footpad swelling was measured at 24, 48 and 72 hours post-challenge. Results were expressed as the difference in swelling (×0.01 mm), before and after antigen boost injection.

Statistical analysis: Results were compared using the Student's t-test and by analysis of variance

### 8.2.2 Results

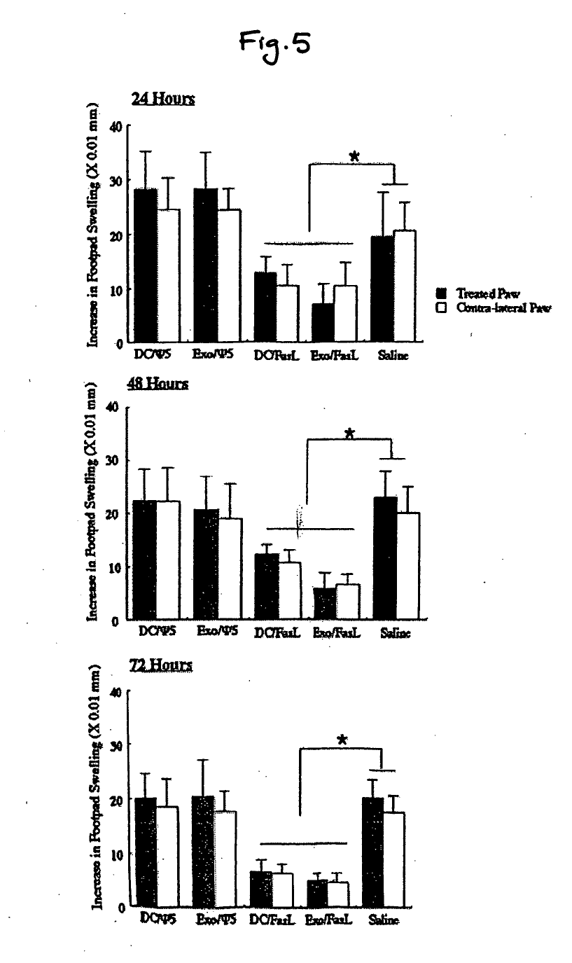

As shown in FIG. 5, delivery of DC/FasL or DC/FasL-derived exosomes significantly suppressed paw swelling in not only in the treated paw, but also in the untreated contralateral paw at 24, 48 and 72 hours post-injection of antigen. In contrast, injection of DC/T5 or exosomes derived from the control DC were unable to inhibit DTH responses. These results demonstrate that genetically modified DC expressing FasL, as well as exosomes derived from the DC/FasL, are equally effective in suppressing the DTH response in not only the treated paw, but also in the contralateral, untreated paw.

The contralateral effect has been observed following both in vivo and ex vivo delivery of a variety of different therapeutic genes in several different animal models of arthritis. (Kim et al., J. Immunol. 164:1576-1581 (2000); Whalen et al., J. Immunol. 162:3625-3632 (1999); Lechman et al., J. Immunol. 163:2202-2208 (1999); Ghivizzani et al., Proc Natl Acad Sci USA. 95:4613-4618 (1998); Kim et al., J. Immunol. 166:3499-3505 (2001); Ijima et al., Hum Gene Ther. 12:1063-1077 (2001); Kim et al., Mol Ther. 6:584-590 (2002); Smeets et al., Arthritis Rheum. 48:2949-2958 (2002); Lechman et al., Gene Ther. 10:2029-2035 (2003)).

### 8.3 Suppression of Delayed Type Hypersensitivity by Syngeneic Exosomes Presenting FasL

### 8.3.1 Materials and Methods

To examine the mechanism(s) through which DC-derived exosomes are able to suppress inflammation, it was examined whether the observed effects were MHC dependent and antigen specific. To determine if the ability of the exosomes to inhibit the DTH response is MHC dependent, in was examined whether allogeneic exosomes were able to suppress the DTH response in vivo. DCs derived from C57BL/6 (H-2b, I-Ab) mouse were used as a source of syngeneic exosomes whereas DCs from Balb/C (H-2d, I-Ad) were used as a source for allogeneic exosomes. To examine the ability of syngeneic and allogeneic DC-derived exosomes to suppress DTH in vivo, KLH-immunized mice were injected with syngeneic or allogeneic exosomes into one of the two hind paws 12 hours prior to injection of antigen into both hind paws. The extent of footpad swelling was measured after a 48-hour period.

### 8.3.2 Results

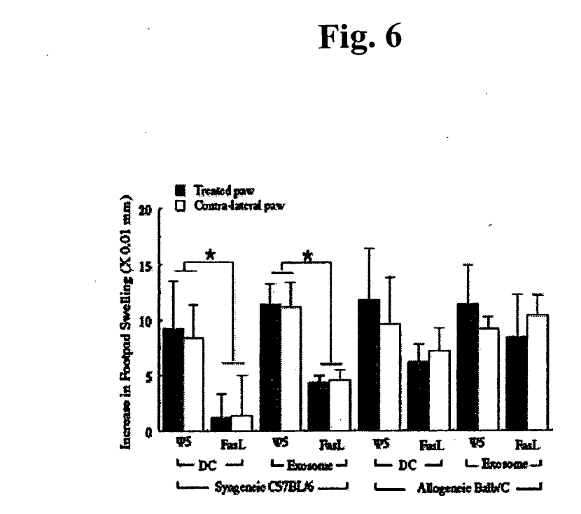

Exosomes derived from allogeneic DC were unable to suppress the DTH response (FIG. 6). In contrast, DTH suppression was observed following injection of either Ad.FasL-transduced DC or DC/FasL-derived exosomes from syngeneic mice. Moreover, local treatment of syngeneic DC/FasL-derived exosomes resulted in reduction of paw swelling both in treated and untreated contra-lateral paws. The in vivo results also demonstrate that the effect of Exo/FasL is not due to wide spread induction of apoptosis from injection of cell membranes carrying FasL.

### 8.4 Delayed Type Hypersensitivity Suppression of Exosomes Presenting FasL is MHC Class II Dependent

### 8.4.1 Material and Methods

To examine further the nature of the immune regulation conferred by the DC and exosomes, DC were prepared from both MHC class I- and class II-deficient mice, and infected with Ad.FasL or Ad.Ψ5. The exosomes were isolated from DC/FasL or DC/Ψ5 of MHC class I- and class II-deficient mice, and injected into the hind paws of KLH-immunized mice.

### 8.4.2 Results

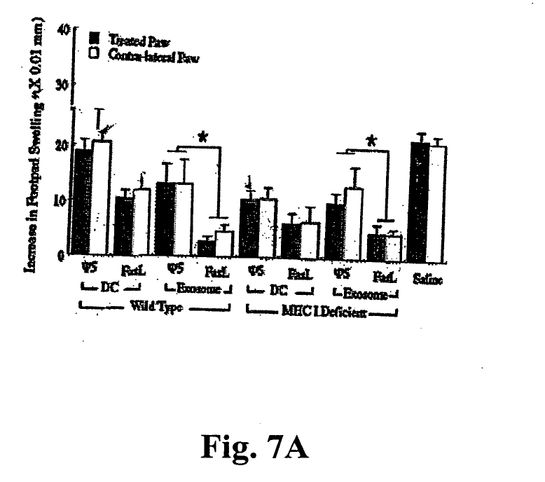

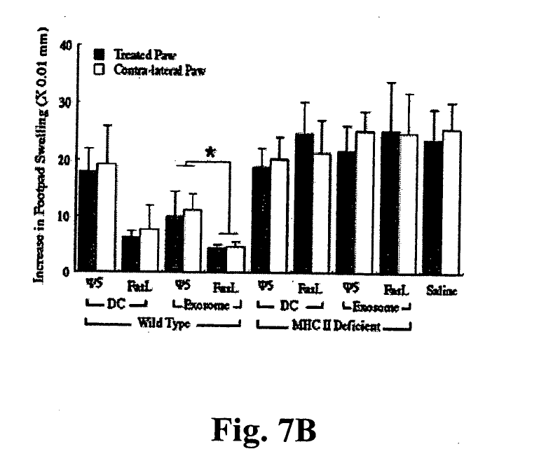

Injection of the genetically modified DC and DC-derived exosomes from class I deficient mice has only a marginal effect on the magnitude of the therapeutic effect (FIG. 7A). However, injection of both FasL expressing DC and DC-derived exosomes from MHC class II deficient mice resulted in complete abrogation of the therapeutic anti-inflammatory effect (FIG. 7B). These results indicate that the therapeutic effect of DC/FasL is MHC class II, but not class I dependent, consistent with a primary role of CD4+ T cells in the regulation of the DTH response (Ptak et al., J. Immunol. 146:469-475 (1991)). In addition, the results indicate that exosomes, similar to DC, also require class II, but not class I, for the observed regulation of DTH response.

### 8.5 The Exosome-Mediated Inhibition of the Delayed Type Hypersensitivity Response is Antigen-Specific

### 8.5.1 Materials and Methods

To demonstrate that exosomes presenting FasL can confer an antigen dependent suppression of the immune response, mice were immunized to KLH. DC were prepared and infected with Ad.FasL or Ad.eGFP and then pulsed with either KLH or Ova protein. Exosomes were prepared from the different DC cultures and injected into the immunized mice just prior to injection of KLH to induce the DTH response, which was measured after 48 hours.

### 8.5.2 Results

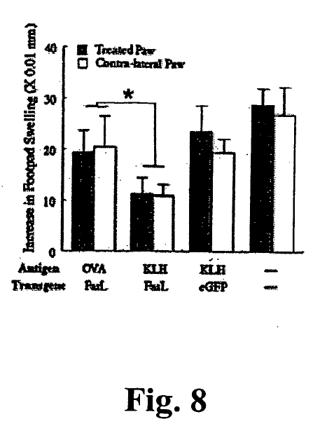

As shown in FIG. 8, exosomes derived from the Ad.FasL infected, KLH pulsed DC reduced inflammation in the injected paw as well as the untreated contralateral paw. In contrast, the exosomes derived from the Ova pulsed, FasL expressing DC were only moderately able to suppress inflammation, similar to the exosomes derived from the KLH treated, Ad.eGFP infected DC. These results suggest that exosomes derived from DC expressing FasL are able to suppress inflammation in an antigen specific manner.

### 8.6 Exosomes Prepared from Dendritic Cells of FasL-Deficient Mice were Unable to Suppress the Delayed Type Hypersensitivity Response

### 8.6.1 Materials and Methods

DC and DC-derived exosomes were isolated from either wild type or gld (FasL−/−) mice and infected with a control adenovirus or with an adenovirus expressing FasL. The DC and DC-derived exosomes were injected back into the footpad of wild type mice, previously immunized to KLH, 12 hours prior to injection of KLH into the footpad. The extent of paw swelling after antigen injection was then measured. The exosomes from the wild type and gld (FasL−/− mice) also were injected back into lpr (Fas−/−) mice to demonstrate that the effect of the exosomes and DC requires the presence of a functional Fas in the recipient mice.

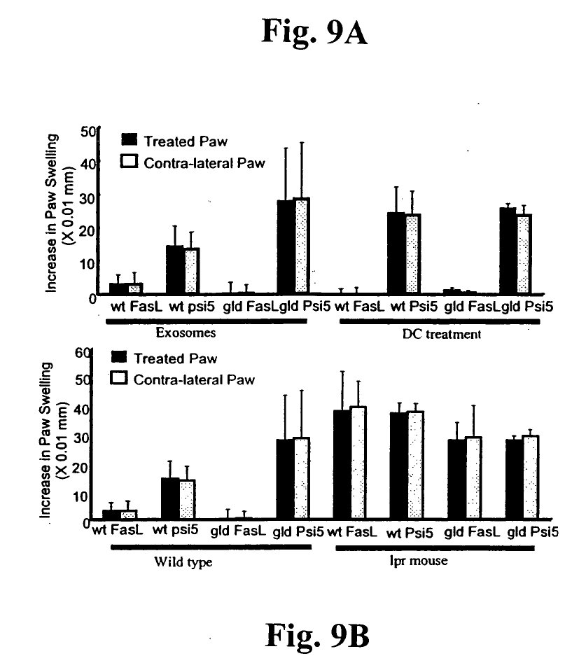

DC and exosomes from gld (FasL−/− mice were unable to suppress the DTH response in the injected and contralateral paw, but the effect was restored if the gld DC and DC-derived exosomes were transduced with Ad.FasL (FIG. 9A). Moreover, the FasL containing exosomes as well as the DC-FasL are ineffective in lpr (Fas−/−) mice (FIG. 9B).

### 8.7 Administration of Exosomes Carrying FasL Ameliorated Disease Severity in Collagen-Induced Arthritis Model

To demonstrate that DC/FasL-derived exosomes can treat collagen-induced arthritis the exosomes were injected into a mouse collagen induced arthritis model.

### 8.7.1 Materials and Methods

Male DBA/1 lacJ (H-2q) mice, 7 to 8 week of age were purchased from the Jackson Laboratory (Bar Harbor, Me.) and maintained in a pathogen-free animal facility at the University of Pittsburgh Biotechnology Center. Bovine Type II collagen (Chondrex) in 0.05 M acetic acid at a concentration of 2 mg/ml was emulsified in an equal volume of Freund's complete adjuvant (FCA) and injected into tail base of the mouse.

The mice were monitored by established macroscopic scoring system ranging from 0 to 4: 0, normal; 1, detectable arthritis with erythma; 2, significant swelling and redness; 3, severe swelling and redness from joint to digit; and 4, maximal swelling and deformity with ankylosis. The average of macroscopic score was expressed as a cumulative value for all paws, with a maximum possible score of 16 per mouse (n=7).

DC were infected with Ad/FasL or Ad/Ψ5 and the exosomes isolated from the infected DC were injected intravenously into DBA 1 mice immunized with bovine type II collagen at day 28.

### 8.7.2 Results

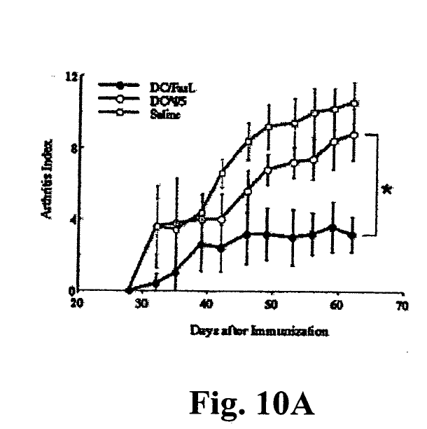

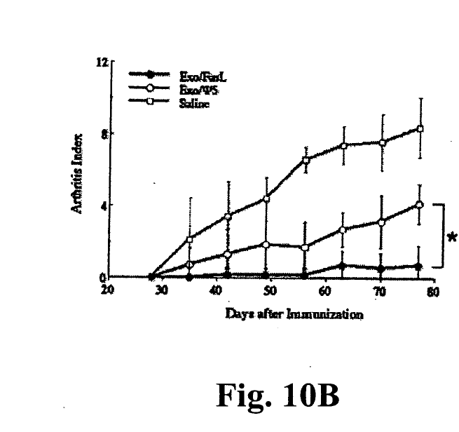

Treatment with DC/FasL (FIG. 10A) and exosomes presenting FasL (FIG. 10B) were able to delay disease onset and suppress the progression of arthritis following a single treatment, whereas the Exo/Ψ5 control group showed an intermediate effect on disease regression compared to DC control and the saline control. These results suggest that a single injection of exosomes derived from DC expressing FasL is able to suppress collagen-induced arthritis. Similarly, injection of Exo/FasL into mice with established disease (on day 32) also suppressed disease.

### 9. EXAMPLE

### Exosomes Derived from DC/IL-10 Have Immunosuppressive Activity

### 9.1 In Vitro Function OF BMDC-Derived Exosomes Presenting IL-10

To demonstrate the ability of BM-DC derived exosomes to suppress T cell proliferation, the effect of adding DC-derived exosomes to a mixed leukocyte reaction (MLR) was examined. As a source of potentially immunosuppressive DC-derived exosomes, BMDC transduced with an adenoviral expressing the Epstein Barr Virus encoded IL-10 gene, termed viral IL-10 (vIL-10) were used. Intra-articular gene transfer of vIL-10 has been shown to suppress inflammation in both rabbit antigen-induced arthritis (AIA) and murine collagen-induced arthritis (CIA) models (5, 6). As a control, BM-DCs transduced with adenoviral vector expressing luciferase (Ad.Luc) were used.

### 9.1.1 Materials and Methods

Vector construction and adenovirus generation: Adenoviruses expressing viral IL-10 (Ad.vIL-10) and enhanced green fluorescent protein (Ad.eGFP) were constructed, propagated, and titered according to standard protocols as previously described (Kim et al., Arthritis Res. 2:293-302 (2000)) Briefly, the recombinant adenoviruses were generated by homologous recombination in 293 cells expressing Cre recombinase (CRE8 cells), after co-transfection of DNA, an adenovirus 5-derived, E1- and E3-deleted adenoviral backbone (psi5) and pAdlox, the adenoviral shuttle vector. The inserted cDNA sequences are expressed under the human CMV promoter. The recombinant adenoviruses were purified by CsCl gradient ultracentrifugation, dialyzed in sterile virus storage buffer, aliquoted and stored at −80° C. until use. The CRE8 cells were grown and maintained in DMEM (Life Technologies, Gaithersburg, Md.) supplemented with 10% fetal calf serum.

Mixed Lymphocyte Reaction:T cells were purified from the spleens of BALB/c mice for in vitro micro-culture in round-bottomed 96-well plates. In each well, 5×10⁴ splenic T cells were seeded with either control C57BL/6 derived DC or genetically modified C57BL/6 derived DC (vIL-10, rIL-10 or Luciferase) or exosomes isolated thereof. DCs were added to the T cells at T cell:DC ratios of 5:1, 10:1, 20:1, and 40:1. On day five of culture, 1 μCi of ³H-thymidine was added to each well 16 h prior to harvest. Radioactive labeling of proliferating T cells was measured on a microplate Beta counter (Wallac, Truku, Finland).

### 9.1.2 Results

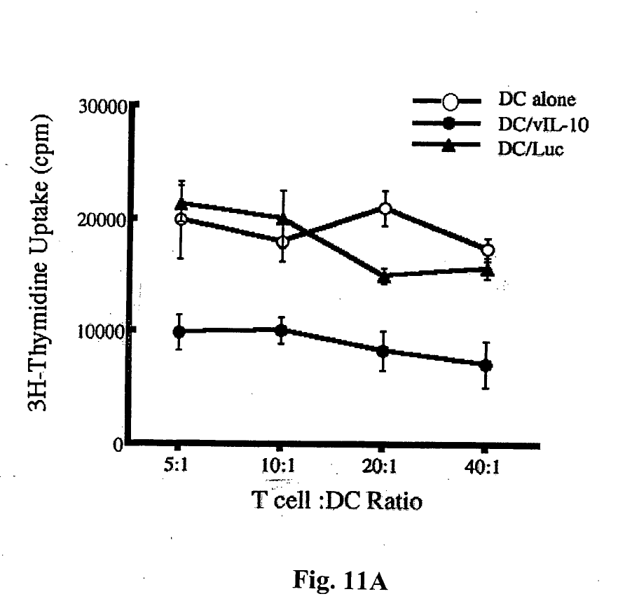

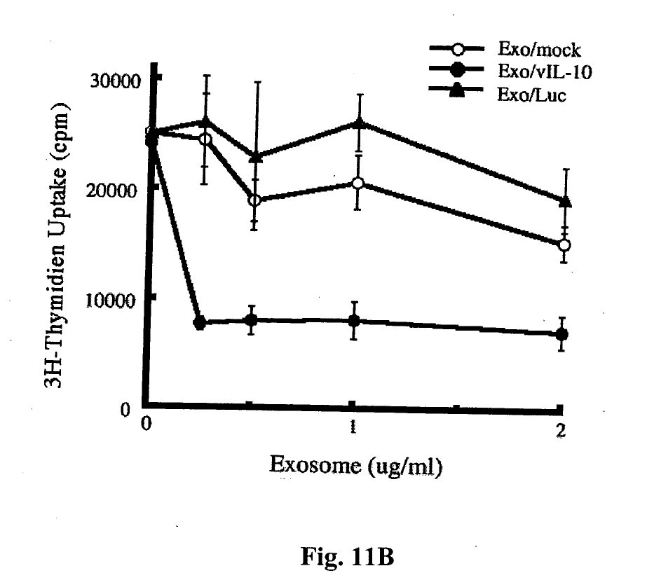

When added to the MLR, the DC transduced with Ad.vIL-10 were able to almost completely suppress T cell proliferation, as measured by 3H Thymidine incorporation, whereas addition of nontransduced DC displayed little or no effect (FIG. 11A). Exosomes secreted by BM-DC infected with Ad.vIL-10 exhibited a moderate four-fold decrease in T cell proliferation (FIG. 11B). These data suggest that exosomes isolated from the immunosuppressive vIL-10 expressing DC are able to block T cell proliferation and that exosomes derived from unmodified BMDC may themselves harbor partial anti-inflammatory properties.

### 9.2 Exosomes Presenting IL-10 Can Suppress Inflammation in a Delayed Type Hypersensitivity Model

To demonstrate the anti-inflammatory effect of BM-DC derived exosomes in vivo, a delayed type hypersensitivity model in C57BL/6 mice was used. This model has been previously used to show that injection of Ad.vIL-10 into one hind footpad results in suppression of inflammation in both the injected and contralateral footpad. Moreover, it has been shown by adoptive transfer experiments that the contralateral effect observed following local Ad.vIL-10 delivery in the DTH model was conferred by endogenous APC. Groups of sensitized mice were injected in the right rear footpad with either untreated or genetically modified DC or the exosomes derived from the culture media of these cells. The contra-lateral footpads received a saline injection of a similar volume. After 12 hours, each footpad was challenged with 20 μg KLH, and footpad swelling monitored at 24, 48 and 72 hours after disease induction.

### 9.2.1 Materials and Methods

Delayed Type Hypersensitivity: On day 0, mice were sensitized by subcutaneous injection of 100 μg antigen (OVA) emulsified 1:1 in Freund's complete adjuvant (Difco, Detroit, Mich.). Two weeks later, pre-sensitized mice were injected in one rear footpad with either 1×10⁶ treated DC (in 50 μl of PBS) or 1 μg purified exosomes derived from each experimental DC group (in 50 μl of PBS). The experimental DC groups include DC transduced with 50 moi of Ad.luciferase and DC transduced with 50 moi of Ad.vIL-10. The contra-lateral footpads were injected with equal volumes of saline. One day later, the mice were challenged in both rear footpads by injecting 20 μg antigen dissolved in 50 μl of PBS and the footpads were measured with a spring loaded caliper (Dyer Co. Lancaster, Pa.) 24 h, 48 h, and 72 h later. The results were expressed as the difference in size due to swelling (mm×10-2).

Statistical Analysis: All data were analyzed using the Microsoft Excel software program. Group comparisons were performed using both student's t test and ANOVA.

### 9.2.2 Results

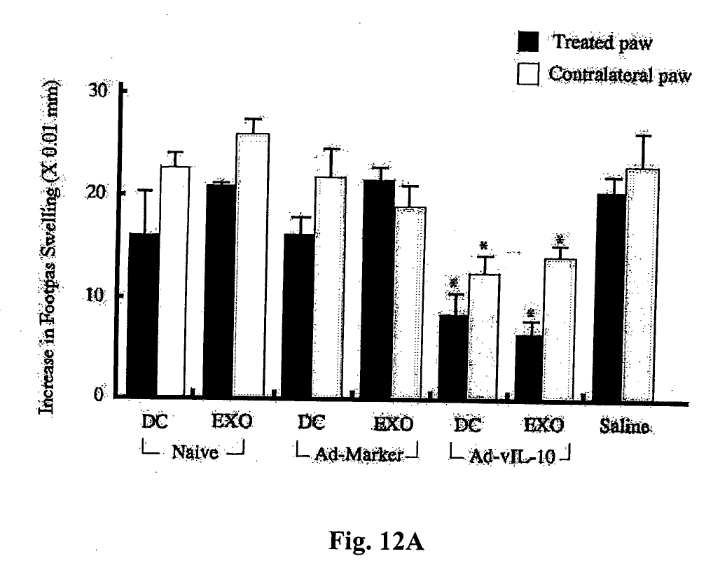

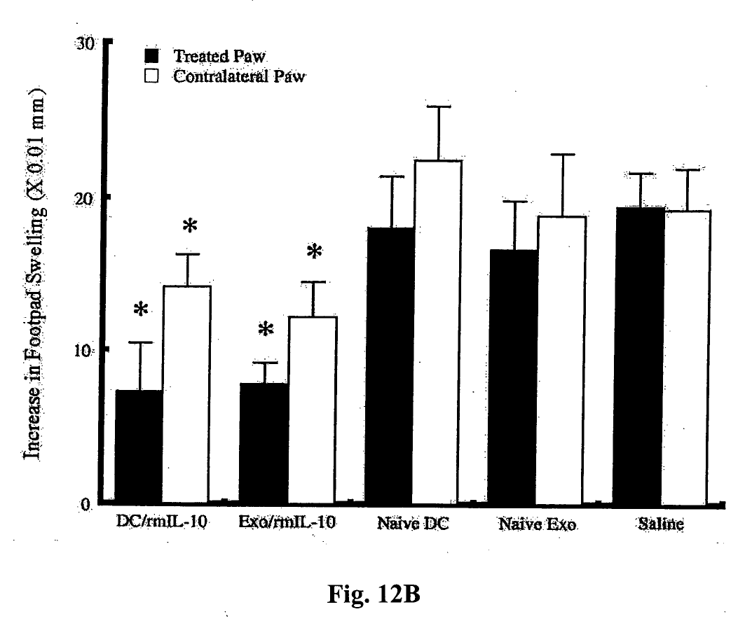

As shown in FIG. 12, the DTH response in saline control animals was acute, with the average increase in paw thickness over 2 mM. However, footpad swelling was reduced by greater than 50% in the injected footpads of mice receiving 1×10⁶ BM-DC transduced with Ad.vIL-10. A reduction of inflammation (40%) was also observed in the saline treated contra-lateral footpads of these same animals. Interestingly, injection of one microgram of secreted exosomes derived from Ad.vIL-10 tranduced BMDC was as even more protective, suppressing paw swelling by 65% compared to saline control mice. Furthermore, a significant reduction was also observed in the footpads contra-lateral to the Ad.vIL-10/exosome treated joints (FIG. 9A). No significant reductions in footpad swelling were observed following administration of Ad.Luc/DC, Ad.Luc/exosomes, untreated DC or exosomes from untreated DC. These data suggest that exosomes derived from Ad.vIL-10 transduced BMDC can suppress DTH in both treated and untreated contra-lateral footpads when delivered locally to sensitized mice.

### 9.3 Recombinant IL-10 Treated DC-Derived Exosomes are Immunosuppressive

### 9.3.1 Materials and Methods

Although the experiments performed above suggest that exosomes derived from Ad.vIL-10 transduced DC are immunosuppressive, it is possible that a low level of Ad.vIL-10 or vIL-10 protein contaminated the exosome preparation. In order to show that adenovirus infection or vIL-10 protein contamination did not contribute to the observed effects, BM-DC were treated with 1 μg/ml recombinant murine IL-10 protein for 24 hours in culture and the generated exosomes tested in vivo utilizing the delayed type hypersensitivity model in C57BL/6 mice as described in Example 9 (FIG. 12B).

### 9.3.2 Results

The exosomes derived from recombinant murine IL-10 treated DC produced a strong immunosuppressive effect 48 hours post-challenge as demonstrated by a six fold reduction in paw swelling in the treated paws, and a 3 fold reduction in the untreated contralateral paws. Taken together, these results demonstrate that exosomes derived from murine IL-10 treated BMDC can suppress DTH in both treated and untreated contra-lateral footpads, effectively ruling out adenovirus contamination as the mechanism for this effect. It is also important to note that no recombinant IL-10 protein was detected in the exosome preparations by ELISA.

### 9.4 Membrane Disruption Causes Loss of Immunosuppressive Ability of Exosomes

### 9.4.1 Materials and Methods

To confirm that the exosomes present in the enriched 100,000×g pellet fraction were important for conferring the therapeutic effects in the DTH model, the requirement for intact exosomes particles for efficacy was examined.

Exosomes from DC or DC transduced with Ad.Luciferase or Ad.vIL-10 were isolated and subjected to four cycles of freeze/thaw. 5 μg of the preparations were separated by SDS-PAGE analysis and blotted for Hsc 70.

To test whether exosomes require an intact membrane to suppress the DTH response, 1 μg of intact or freeze/thaw injected exosomes from DC transduced with Ad-vIL-10 were treated into one hind footpad of KLH-immunized mice (FIG. 10C). After 24 hours, each footpad was boosted with KLH and the extent of footpad swelling measured.

### 9.4.2 Results

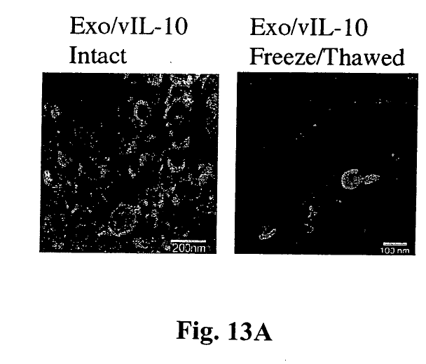

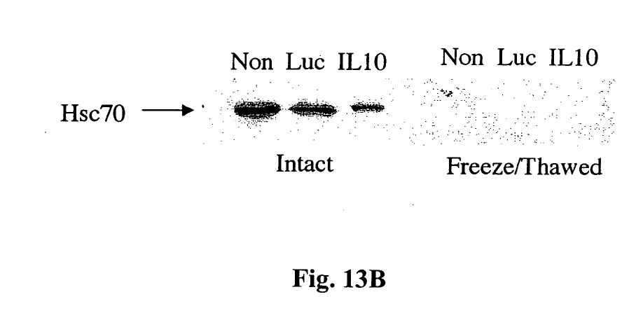

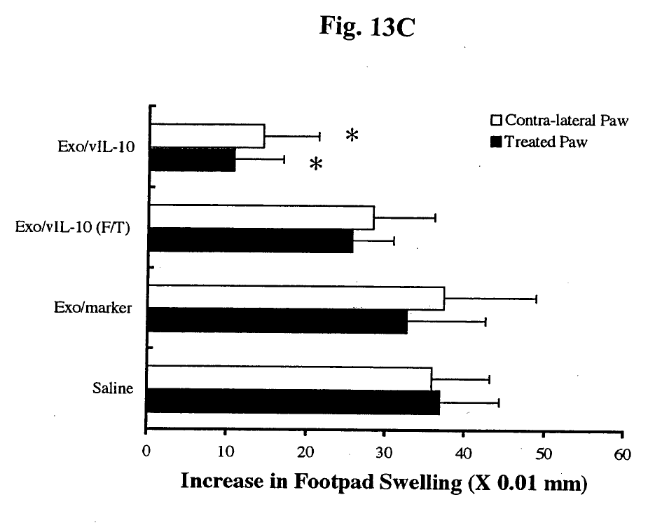

The fact that the integrity of the exosomes was disrupted by 4 cycles of freeze/thaw was demonstrated by electron microscopy (FIG. 13A). Indeed, the soluble, exosome-associated protein Hsc 70 was not detected in the freeze/thaw treated exosome fractions whereas it was associated with untreated exosomes (FIG. 13B).

Whereas reduction of footpad swelling was observed in the group treated with intact exo/vIL-10, the group injected with freeze/thaw treated exo/vIL-10 showed no reduction in paw swelling, similar to the control groups treated with saline or exosomes from control DC. These results demonstrate that multiple cycles of freeze/thaw disrupted membrane structure of exosomes thereby eliminating the immunosuppressive ability of exosomes in the DTH model.

### 9.5 MHC Class II-Containing Exosomes Required for Suppression of the Delayed Type Hypersensitivity Response

The data below demonstrates the effect of depleting MHC class II positive exosomes on suppression of the DTH response was examined.

### 9.5.1 Materials and Methods

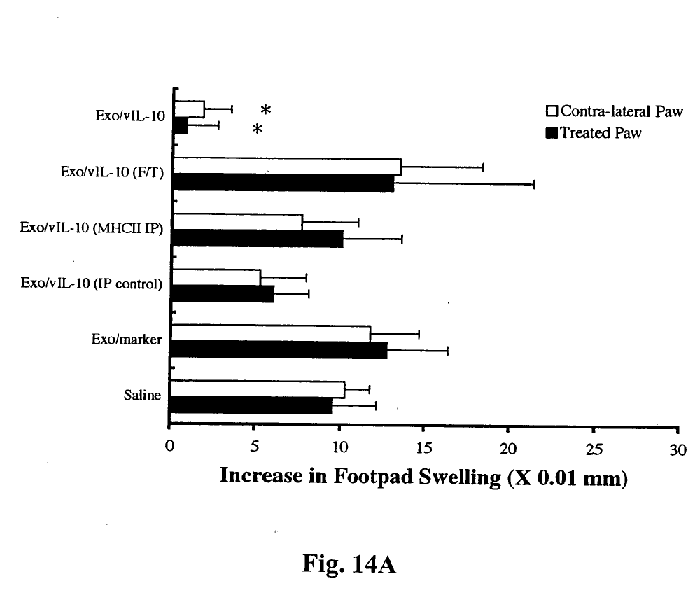

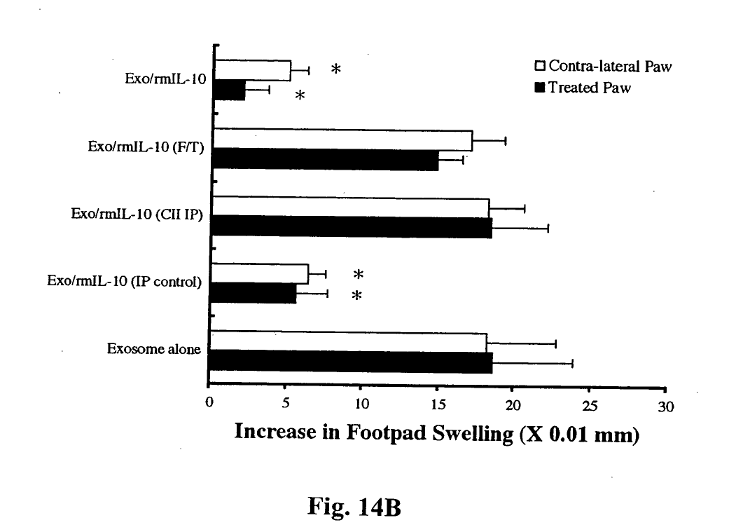

Exosomes from Ad.vIL-10-transduced BMDC were divided into four samples for pre-treatment prior to injection into sensitized mice (FIG. 14A). The first exosome sample was pre-adsorbed with paramagnetic beads specific for murine MHC II (Exo/vIL-10(MHC II IP)), whereas the second sample was pre-adsorbed with paramagnetic beads specific for NK1.1 (Exo/vIL-10(IPcontrol)), a cell surface molecule not present on DC-derived exosomes. The third sample was subjected to multiple cycles of freeze/thaw (Exo/vIL-10(F/T)), whereas the fourth sample was left untreated. The exosome samples were then injected into one hind footpad of mice and 12 hours later, DTH induced footpad swelling in both hind footpads was measured over the next 72 hours.

### 9.5.2 Results

Exosomes from control DC have no effect on footpad swelling whereas Ad.vEL-10/exosomes were able to dramatically block DTH in both the injected and untreated contra-lateral footpads. The exosome preparation preadsorbed to the NK1.1 beads exhibited immunosuppressive activity. However, pre-adsorption of the Ad.vIL-10/exosome sample with class II beads abrogated almost 100% of the activity in vivo. Importantly, the injection of the actual paramagnetic beads with bound class II positive exosomes resulted in immunosuppressive activity similar to that seen with the with the non-adsorbed Ad-vIL-10 exosomes. Exosomes from recombinant mouse IL-10 protein-treated DC showed similar results in the DTH response after MHC II depletion (FIG. 14B). The results were consistent with each other. The formulation that underwent several freeze/thaw cycles in both the exosomes from Ad-vIL-10- or recombinant IL-10-treated DC also lost activity (FIG. 13C). These data suggest that the in vivo anti-inflammatory effects seen with the administration of the exosome fraction to mice are MHC class II dependent. Since MHC class II is present on the exosome surface at high levels, this suggests that the in vivo activity seen in mice is attributable to exosomes. Furthermore, it appears that the integrity of the vesicle is also required. These in vivo anti-inflammatory effects also appear to be specific for structurally intact MHC class II positive exosomes, effectively ruling out adenovirus contamination as the mechanism for the effect.

### 9.6 Exosomes Presenting IL-10 Can Suppress Collagen-Induced Arthritis in Mice

### 9.6.1 Materials and Methods

Bovine type II collagen (Chondrex L.L.C., Redmond, Wash.) was dissolved in 0.05 M acetic acid at a concentration of 2 mg/ml by stirring overnight at 4° C. and was emulsified in an equal volume of Freund's complete adjuvant (CFA). The mice were immunized intradermally at the base of the tail with 100 μg collagen. On day 21 after priming, the mice received an intradermal booster injection of Type II collagen in Freund's incomplete adjuvant (IFA). Mice were monitored every other day by an established macroscopic scoring system ranging from 0 to 4: 0=normal; 1=detectable arthritis with erythema; 2=significant swelling and redness; 3 severe swelling and redness from joint to digit; and 4=maximal swelling and deformity with ankylosis. The average of macroscopic score was expressed as a cumulative value for all paws, with a maximum possible score of 16 per mouse. The thickness of each paw was also measured with a spring-loaded caliper and the paw swelling for each mouse was calculated by adding the thickness of all four paws. The in vivo experiments were performed with 10 mice per group and repeated twice to ensure reproducibility.

Rheumatoid arthritis (RA) is a debilitating autoimmune disease characterized by chronic inflammation of the distal diarthroidial joints and progressive destruction of cartilage tissue. Similar pathologies as well as inflammation in joints can be induced in the DBA1/lacJ (H-2kq) strain with injection of bovine type II collagen. To examine the ability of DC and DC-derived exosomes to treat collagen-induced arthritis, DC were infected with Ad-vIL-10 and the resultant exosomes were and then injected intravenously into DBA1 mice immunized with Bovine Type II collagen. Injection was done at day 28, just prior to disease onset.

### 9.6.2 Results

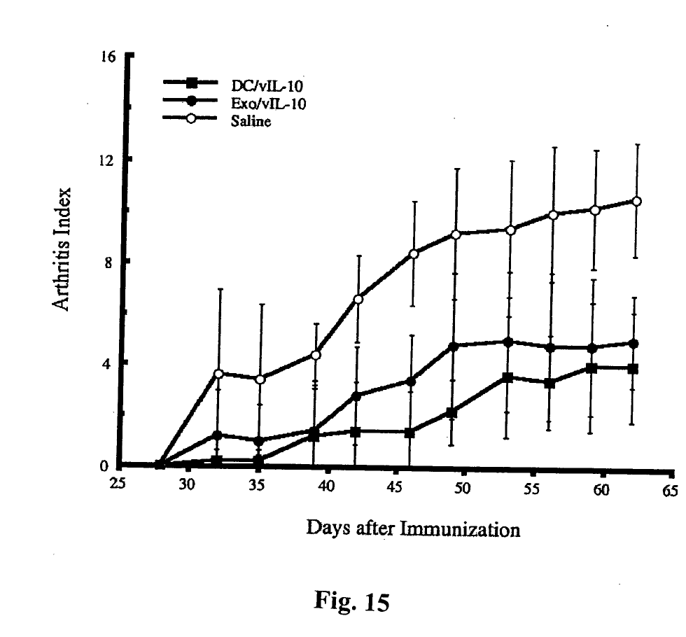

A single injection of either DC/vIL-10 or exosomes from DC/vIL-10 was able to delay the onset and reduce the severity of arthritis, while disease progressed normally in the saline injected control group (FIG. 15). This result indicates that a single injection of exosomes derived from DC expressing IL-10 is comparable to an injection of genetically modified DC in conferring able to confer a therapeutic effect systemically in an autoimmune, inflammatory disease.

In addition to the analysis in DC-derived exosomes in the prevention of disease study, exosomes from DC-IL-10 were tested in the mice with established CIA. Exosomes from Ad.vIL-10-transduced or recombinant IL-10-treated DC were injected intravenously into the mice with established disease (FIG. 16A-C). Even though disease suppression in exo-IL-10-treated group was less than one shown in the prevention study, exosomes from both the Ad.vIL-10 transduced and recombinant IL-10-treated DC were able to reduce the severity of established disease (FIG. 16A). Moreover, freeze/thaw treatment of the exosomes abrogated the therapeutic effect (FIG. 16B) whereas direct injection of recombinant IL-10 had no effect on disease progression (FIG. 16C).

### 10. EXAMPLE

### DC/IL-4 Derived Exosomes Have Immunosuppressive Activity

### 10.1 DC/mbmIL-4 and Exosomes Prepared Therefrom Suppress Delayed Type Hypersensitivity Response

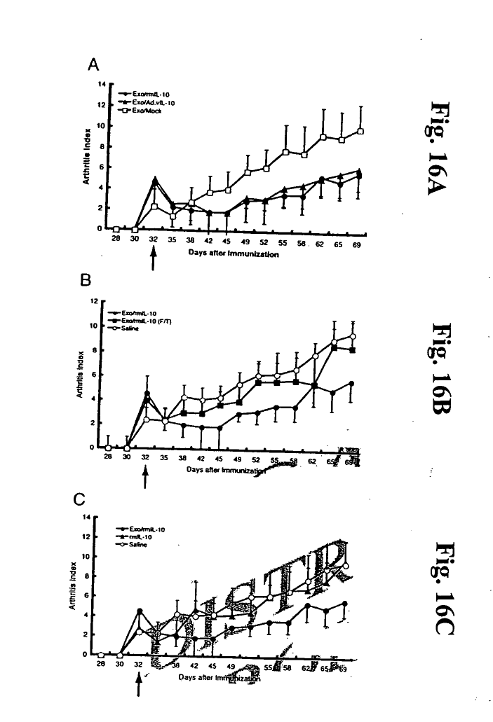

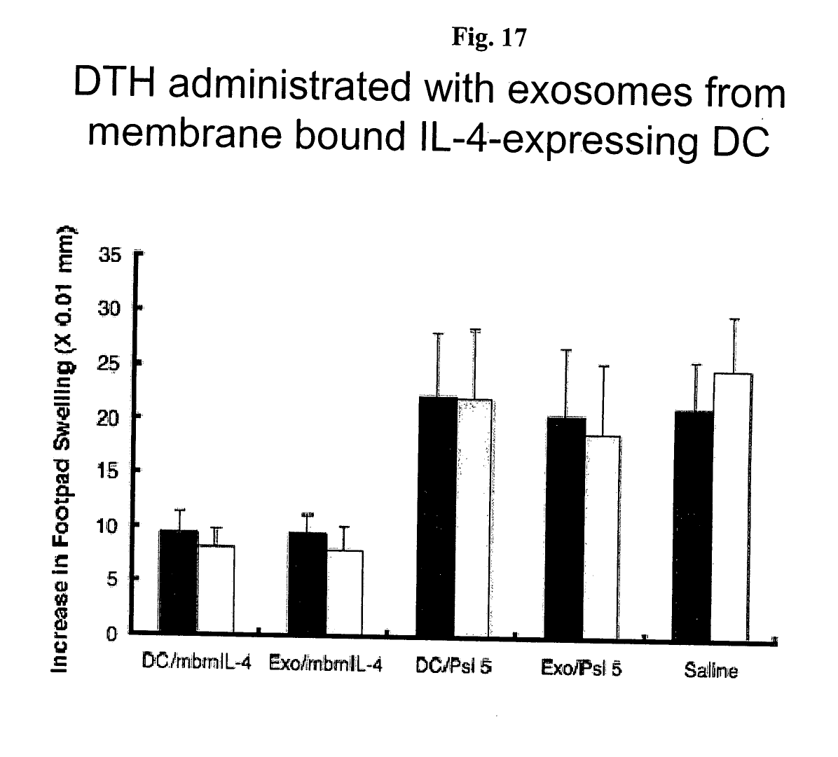

Dendritic cells were transfected with either an adenoviral vector carrying a membrane-bound form of IL-4 (Ad.mbmIL-4) or a negative control virus (Ad.Psi5 or Ψ5). From a portion of these DCs, exosomes were prepared. DCs and exosomes were then tested in the mouse footpad DTH model described herein. The results are shown in FIG. 17. Both DC/mbmIL-4 and exosomes prepared from DC/mbmIL-4 suppressed the DTH response in both the injected paw and its contralateral paw, demonstrating the effectiveness of membrane-bound IL-4 as an enhancing agent.

### 10.2 DC/smIL-4 and Exosomes Prepared Therefrom Suppress Delayed Type Hypersensitivity Response

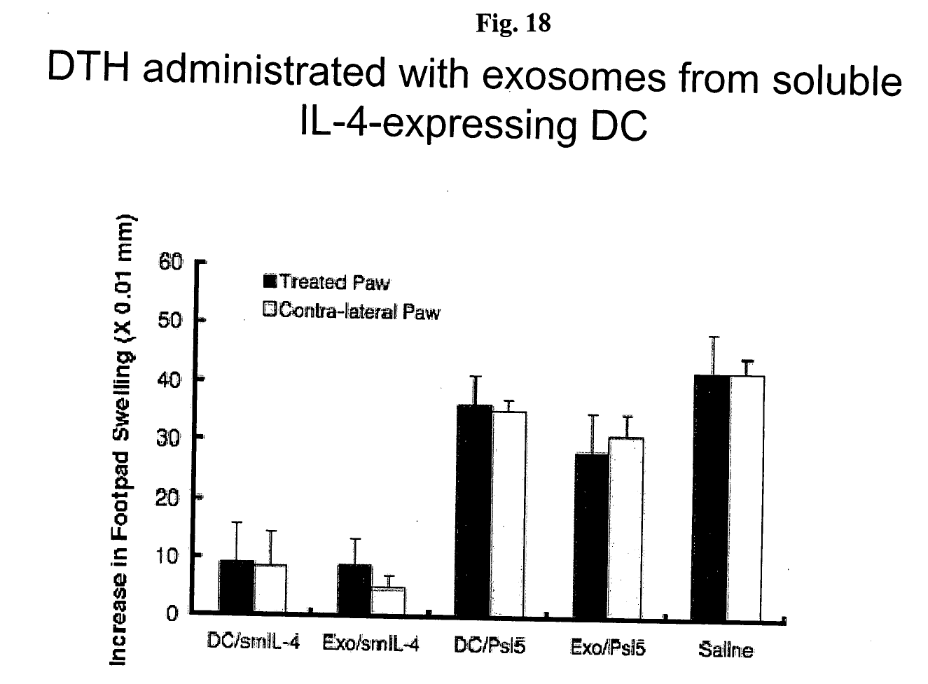

Dendritic cells were transfected with either an adenoviral vector carrying a soluble form of IL-4 (Ad.sIL-4) or a negative control virus (Ad.Psi5 or Ψ5). From a portion of these DCs, exosomes were prepared. DCs and exosomes were then tested in the mouse footpad DTH model described herein. The results are shown in FIG. 18. Both DC/smIL-4 and exosomes prepared from DC/smIL-4 suppressed the DTH response in both the injected paw and its contralateral paw, demonstrating the effectiveness of soluble IL-4 as an enhancing agent.

### 10.3 DC/mbmIL-4 and Exosomes Prepared Therefrom as Well as DC/FasL and Exosomes Prepared Therefrom Inhibited the Delayed Type Hypersensitivity Response

### 10.3.1 Materials and Methods

Exosomes were isolated from C57/BL6 bone marrow derived DC that were either uninfected or infected with an Ad.eGFP control vector, Ad.FasL or an adenoviral vector carrying a membrane-bound form of IL-4 (Ad.mbIL-4). The purified exosomes (1 μg of total protein) as well as the different DC populations (5×10⁵ cells) were injected into the right footpad of mice that had been pre-immunized to KLH. 24 hours post-injection of exosomes or DC, KLH antigen was injected into the right and left rear footpads and the extent of swelling measured over a 48 hour period for both the treated footpad and its left, contralateral footpad.

### 10.3.2 Results

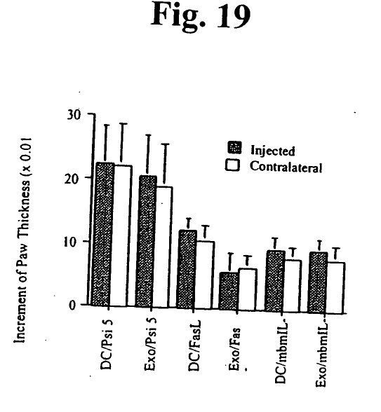

To determine if the exosome fraction, derived from FasL or IL-4 modified DC, was effective in inhibiting the DTH response, DC were isolated from C57/BL6 mice and genetically modified by adenoviral infection to express FasL or IL-4. For these experiments, a membrane-bound version of IL-4 containing the membrane-spanning region of CD28 fused to IL-4 was used (mbmIL-4). The DC and exosome fractions were then injected into the right read footpad 24 hours prior to injection of antigen into both rear footpads. Interestingly, both the DC and the exosome fraction from Ad.FasL and Ad.mbmIL-4 infected DC were able to suppress the inflammatory response not only in the treated paw, but in the contralateral paw as well (FIG. 19). This result suggests that exosomes and the parental DC are able to confer systemic immunosuppression following local injection through what is an antigen-specific mechanism. Without being bound to any particular theory, exosomes may interact in vivo with antigen presenting cells in the draining lymph nodes that are processing the specific antigen, resulting in antigen-specific suppression.

### 10.4 DC/IL-4 Derived Exosomes Require FasL and Fas

DC were isolated from wild type and gld (FasL−/−) mice and modified to express either soluble (smIL-4) or membrane-bound (mbmIL-4) murine IL-4. The DC populations and the DC-derived exosomes were injected back into the footpad of either wild type of lpr (Fas−/−) mice and the effect on paw swelling measured following antigen injection into the footpad.

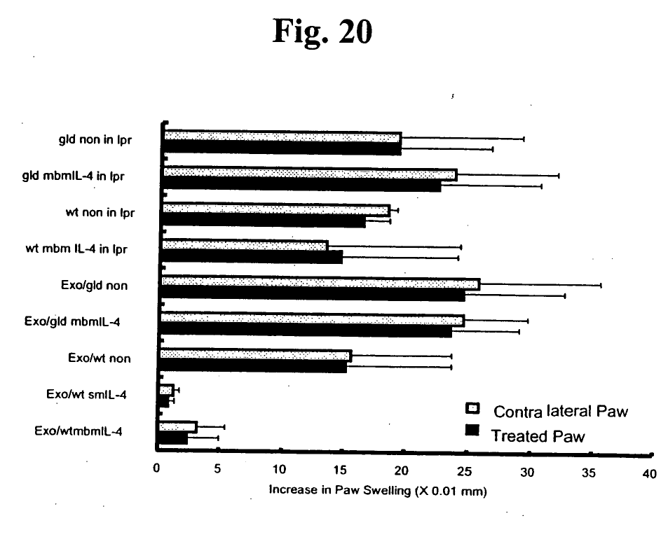

The exosomes isolated from DC/IL-4 (either membrane bound or soluble) require FasL as well as Fas in the recipients to suppress the DTH response (FIG. 20).

### 10.5 Syngeneity is Required for Exosome Immunosuppression

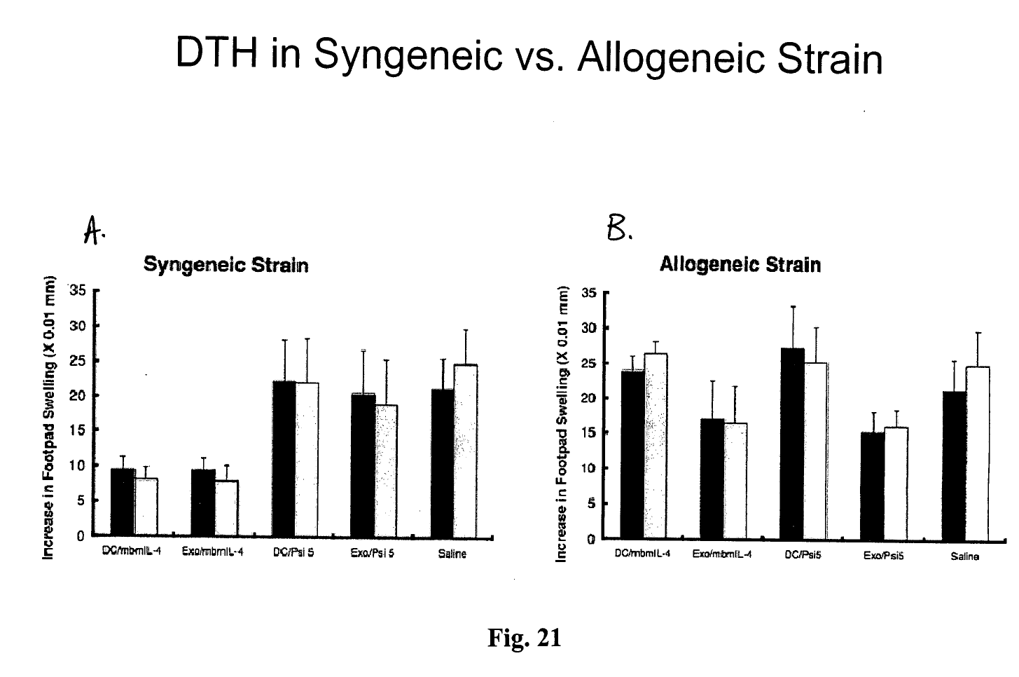

As shown in FIG. 21A-B, only exosomes prepared from syngeneic, but not allogeneic, DC/mbmIL-4 were observed to suppress the DTH response in a murine footpad model, consistent with a role of MHC Class II antigens in exosome-mediated immunosuppression.

### 10.6 DC/IL-4 Derived Exosomes Require B7.1 and B7.2

### 10.6.1. Materials and Methods

DC were isolated from mice deficient in both B7.1 and B7.2 (KO) and infected with either a control (Psi-5) or IL-4 expressing adenoviral vector. Exosomes were isolated from the different DC populations that were then infected (1 μg of exosomes) into one paw on KLH immunized mice. The extent of paw swelling was measured in injected as well as untreated paws.

### 10.6.2 Results

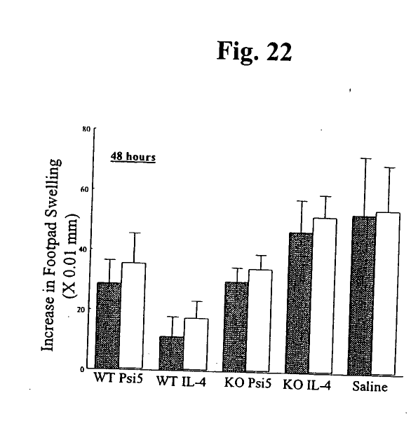

As shown in FIG. 22, exosomes derived from wild type DC modified to express IL-4 by adenoviral gene transfer were able to suppress the DTH response whereas exosomes derived from B7.1/B7.2 double GKO (“KO”) DC had no effect on the DTH response. In addition, the B7 deficient DC had no effect on the DTH response, similar to results observed with B7 deficient DC-derived exosomes. A similar requirement for B7 for induction of T regulatory cells for suppression of diabetes has been observed by Lohr et al., 2003, Nature Immunol. 4:664.

Further, it has been observed that immunosuppressive DC and DC-derived exosomes stimulate a CD4+ CD25+ population of T cells that may represent a regulatory T cell population.

### 10.7 Exosomes Derived from DC/IL-4 Ameliorate Arthritis in a Collagen-Induced Arthritis Model

### 10.7.1 Materials and Methods

Exosomes were isolated from DBA1 bone marrow derived DC that were either mock infected or infected with either Ad.psi-5 (control), Ad.mIL-4, or Ad.mbmIL-4. The exosomes (1 μg of total protein) were then injected intravenously into DBA1 mice after disease onset (day 32). The severity of arthritis was monitored for each paw (scale 0-4 with a maximum score of 16) for each of the treatment groups for a one month period.

### 10.7.2 Results

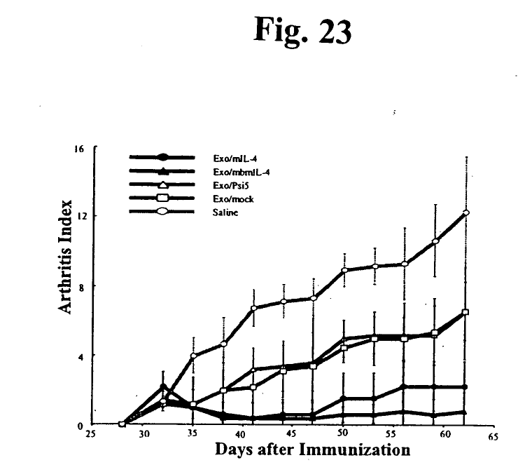

As shown in FIG. 23, treatment with exosomes prepared from DC/IL-4 and in particular from DC/mbmIL-4 were able to block the progression of disease (at day 32, all of the test animals showed signs of disease pathology). Some of the treated mice appeared to be disease free.

### 11. ADOPTIVE TRANSFER OF IMMUNOSUPPRESSIVE ACTIVITY

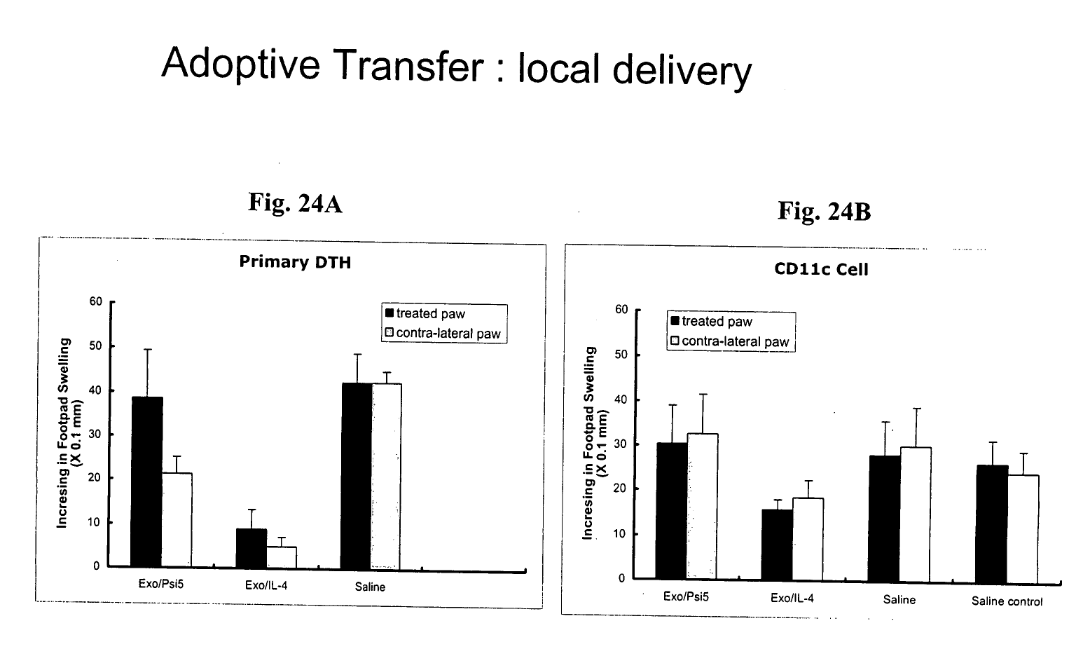

As shown in FIG. 24A-B, adoptive transfer of CD11C cells from mice treated with exosomes can block a DTH response, demonstrating that CD11C positive cells are a target for regulation by the immunosuppressive exosomes.

### 12. EXOSOME-MEDIATED SUPPRESSION OF HYPERGLYCEMIA IN A MURINE MODEL FOR DIABETES

### 12.1 Materials and Methods

DC were generated from the bone marrow of young NOD mice and either infected with Ad.IL-4 or not infected. The exosomes from the DC populations were isolated and 1 μg of exosomes were injected intravenously into 5-6 week old NOD mice with sale treatment used as a control. The animals were then monitored for hyperglycemia.

### 12.2 Results

Based on the significant therapeutic effects observed in the CIA mouse model, the ability of immunosuppressive DC has also been tested for activity in blocking the onset of hypoglycemia. Intravenous injection of DC derived from the bone marrow of young NOD mice (3-4 weeks of age), modified to express IL-4, has been observed to reduce the percentage of NOD mice that become hyperglycemic if administered at 10 weeks of age. Moreover, it has been shown that treatment of bone marrow derived DC from NOD mice, treated with an NF-κB inhibitor (a double-stranded NF-κB decoy oligonucleotide), which results in a more immature DC phenotype (CD80, CD86 and CD40 low), is able to suppress the onset of hyperglycemia in NOD mice. Consistent with these observations as well as results in the CIA model, DC genetically modified to express FasL have been shown to suppress the onset of hyperglycemia in NOD mice if administered during the early stages of insulitis (J. Mountz). Thus, immunosuppressive DC, modified to express either IL-4 or FasL, or treated with an NF-κB inhibitor, may be able to prevent or reverse disease.

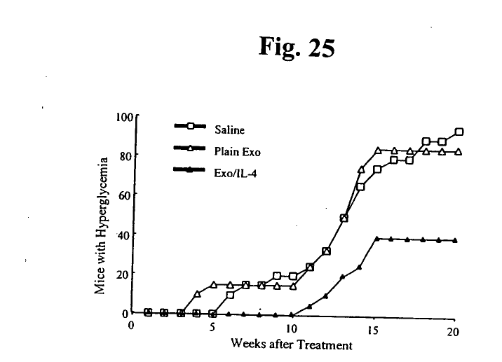

As shown in FIG. 25, in the experiments set forth above, exosomes from Ad.Il-4 transduced DC were able to delay as well as reduce the frequency of the onset of hyperglycemia in NOD mice.

### 13. EXAMPLE

### Exosomes Prepared from Serum

### 13.1 Application of Serum Suppresses the DTH Response

### 13.1.1 Materials and Methods

C57BL6 mice were immunized with an intra-dermal injection of KLH. Two weeks later, blood was collected from the mouse. The sample was centrifuged at 1500 g for 10 minutes to isolate the serum. A total of 50 μl of serum was injected into a hind paw of KLH immunized mouse 24 hours prior to the boost injection of KLH antigen into both hind paws. Footpad swelling was measured 48 hours after KLH-boost injection.

### 13.1.2 Results

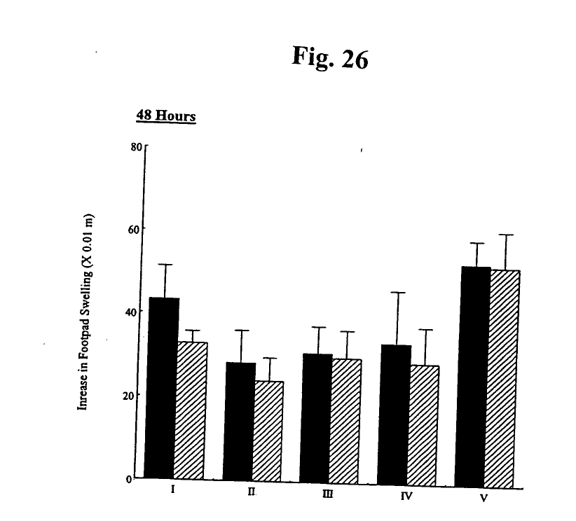

Injection of serum from a naive mouse, incubated with beads for 24 hours showed some reduction in swelling compared to the saline-treated control (See FIG. 26, Group I). Injection of serum from the KLH-immunized mouse, without incubation or beads showed the largest reduction in footpad swelling compared to the saline-control (See FIG. 26, GROUP II). Injection of serum from the KLH-immunized mouse, with incubation both without and with Minikin, which is a small syringe appropriate for the amount of serum that can be isolated from humans and mice, showed some reduction in swelling compared to the saline-treated control (See FIG. 26, Groups III and IV).

### 13.2 Exosomes from Serum Suppress the DTH Response

### 13.2.1 Materials and Methods

A mouse was immunized with an intra-dermal injection of KLH. Two weeks later, blood was collected from the mouse and iced for 4 hours. The sample was centrifuged at 1500 g for 10 minutes to isolate the serum. The exosomes were then isolated by differential centrifugation. Collected serum was centrifuged at 1500 g for 20 minutes and 3000 g for 30 minutes. The supernatant from the final centrifugation was centrifuged again at 100,000 g for one hour in an ultra-centrifuge. The exosome pellet was washed in saline, centrifuged at 100,000 g for one hour, and resuspended in saline.

One microgram of exosomes in 50 micro liter of saline was injected into a footpad of a hind paw of a KLH immunized mouse. The other hind paw was given the same amount of saline. Twenty-four hours later, both hind paws of the mouse were injected with 20 micro gram of KLH antigen in 50 micro liter of saline. Footpad swelling was measured 48 hours after KLH-boost injection.

Briefly, recovered culture supernatant from each BMDC culture was subjected to three successive centrifugations at 300 g (5 min), 1,200 g (20 min), and 10,000 g (30 min) to eliminate cells and debris, followed by centrifugation for 1 h at 100,000 g. To remove excess serum proteins, the exosome pellet was washed with a large volume of PBS and centrifuged at 100,000 g for 1 h, and finally resuspended in 120 μl of PBS for further studies.

To obtain the microvesicles, serum was spun 3,000 g (20 min) and 10,000 g (30 min) and resuspend the pellet with saline. The supernatant of serum was obtained after ultra-centrifugation excluding exosome pellet. The supernatant was not the subject of the further centrifugation.

### 13.2.2 Results

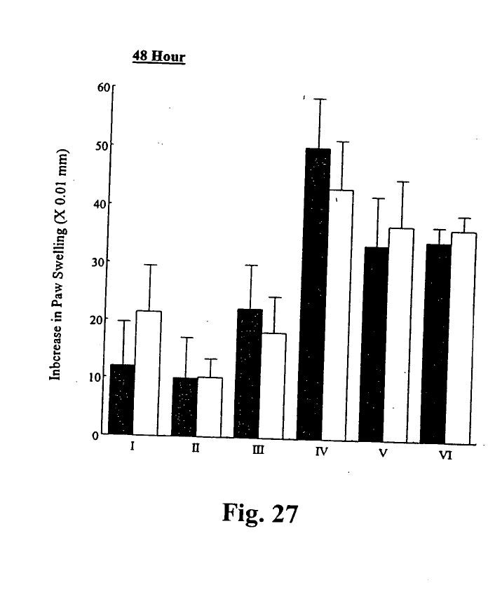

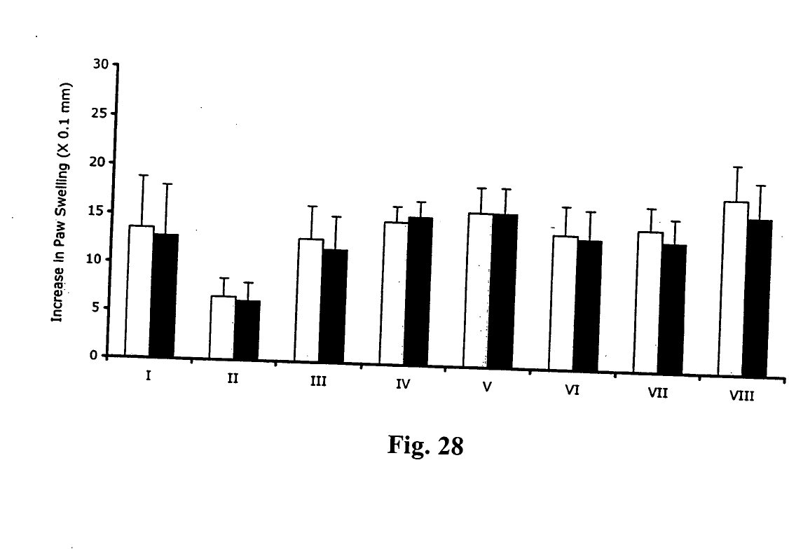

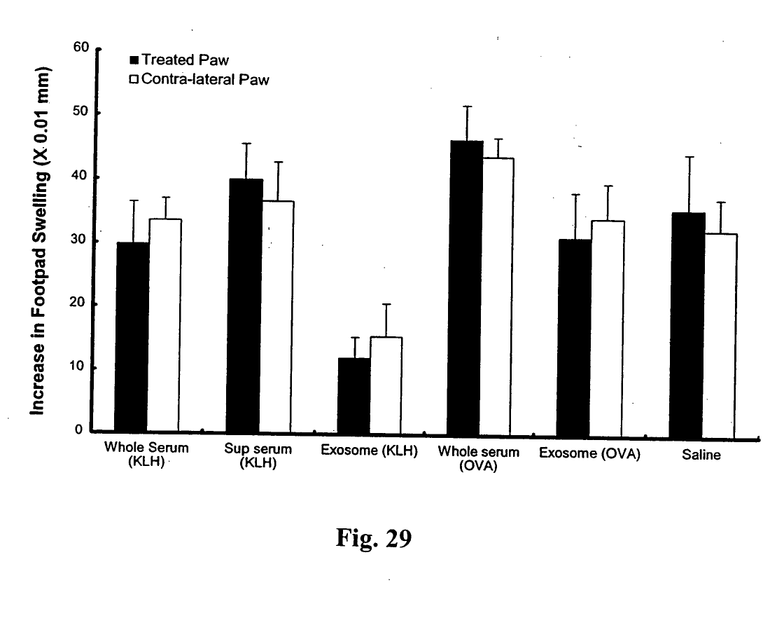

Administration of microvesicles of serum, exosomes of serum, and whole serum from a KLH-immunized mouse produce a reduction of swelling compared to administration of the saline-treated control or serum from a naive mouse to the DTH mouse (FIG. 27). Exosomes produce the greatest reduction of swelling, with significantly more reduction than the use of whole serum (FIG. 27, Group II). Exosomes of serum from a KLH-immunized mouse caused a greater reduction in swelling than the supernatant, freeze/thawed exosomes, and sonicated exosomes from a KLH-immunized mouse and much greater reduction than the supernatant and exosomes from a non-immunized mouse and the saline-treated control (FIG. 28). Group VII is treated with whole serum from the non-immunized mouse. Group VIII is the control group treated with saline. Administration of exosomes of serum from a KLH-immunized mouse showed much greater reduction in swelling than exosomes of serum from an OVA-immunized mouse (FIG. 29).

### 13.3 Serum-Derived Exosomes Suppress a Delayed Type Hypersensitivity Response in an Antigen-Specific, MHC Class II-Dependent Manner

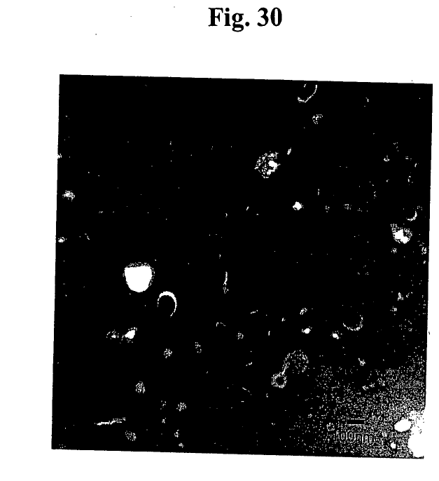

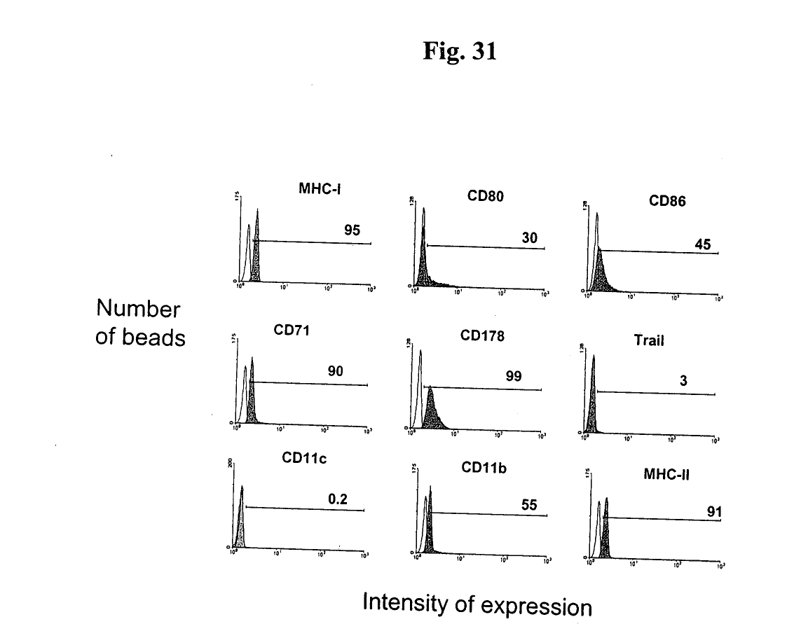

FIG. 30 is an electron micrograph showing exosomes purified from mouse serum. FIG. 31 is a FACS analysis of serum-derived exosomes labeled with beads carrying anti-MHC class II antibodies. Note that all the class II positive exosomes were found to be positive for CD178 (FasL).

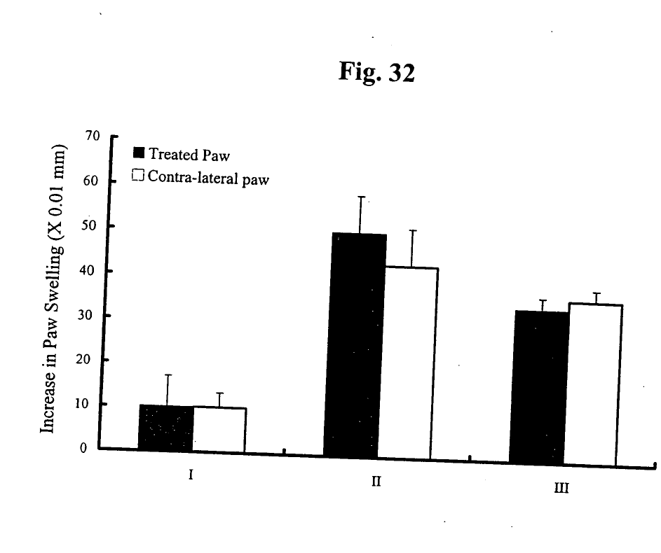

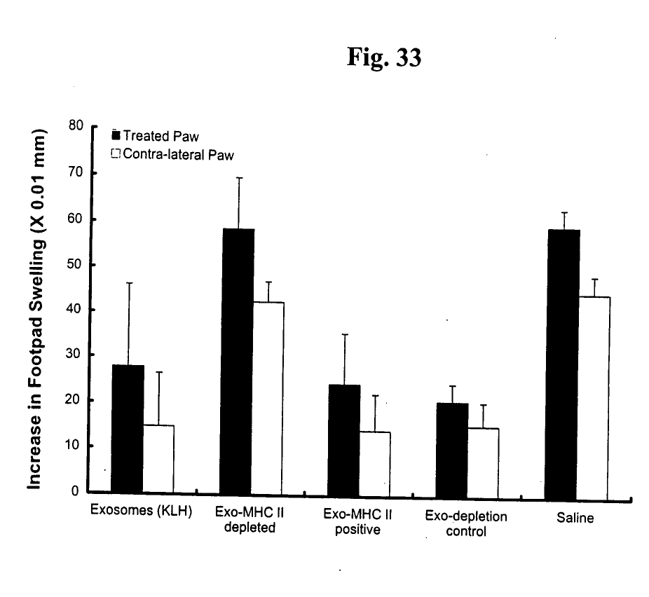

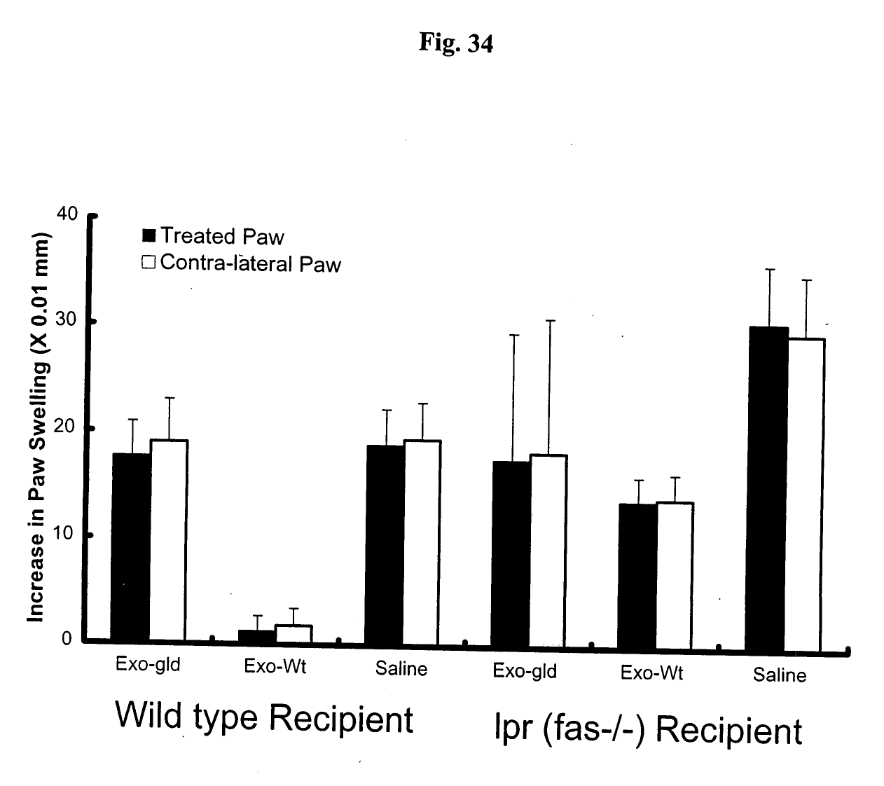

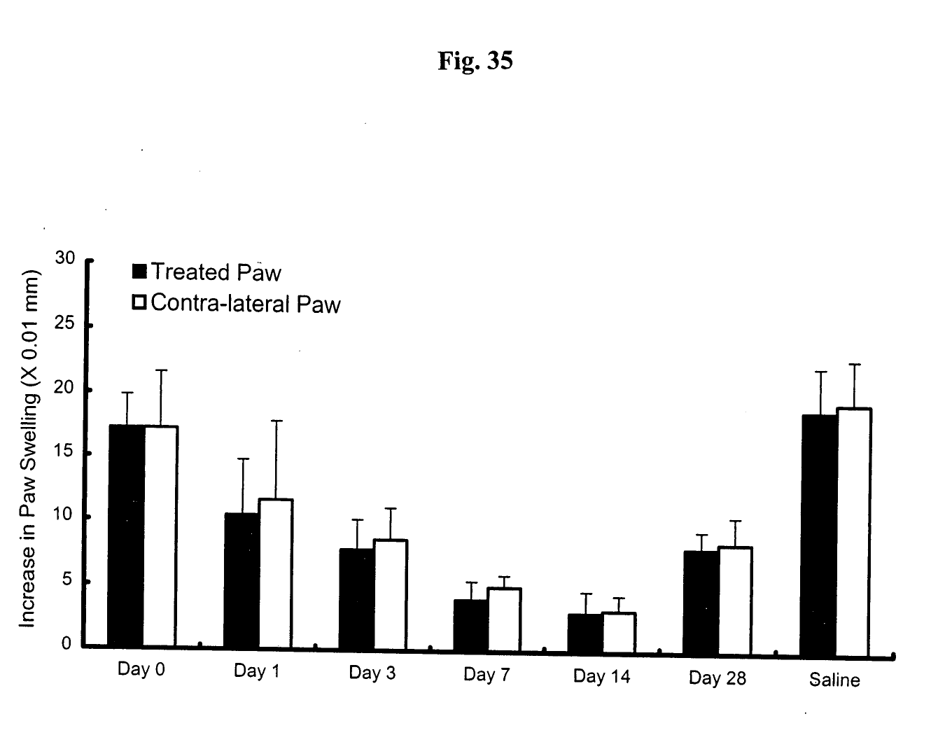

As shown in FIG. 32, exosomes prepared from serum of mice immunized against KLH were effective in suppressing DLH to KLH, but exosomes prepared from serum of naive mice were not, illustrating antigen specificity. FIG. 33 demonstrates that a serum-derived exosome preparation depleted for exosomes carrying MHC class II antigens lost essentially all immunosuppressive activity, illustrating MHC class I dependence of serum-derived exosomes. FIG. 34 illustrates that the immunosuppressive effect of serum-derived exosomes peaks 14 days after immunization with antigen.

### 14. EXAMPLE

### Exosomes Prepared from Serum are Immunosuppressive in Human Subjects

### 14.1 Interleukin-1 Receptor Antagonist-Continued Serum (Orthokine®) Reduces Lumbar Radicular Pain

A randomized, double-blind clinical study was performed to evaluate the effect of IL-1R Antagonist conditioned serum in reducing lumbar radicular pain in 84 patients. The data indicates that exosomes derived from such conditioned serum may be used to reduce the sensation of pain.

### 14.1.1 Materials and Methods

Bead preparation: Blood was incubated in either microtiter plates (24 and 48 wells, Nunc, Denmark) or 60 ml syringes (Perfusor Syringes, Becton Dickinson, USA). The syringes contained 200 glass beads. Glass beads were 2.5 mm in diameter, had a surface area of 21 mm² and were of medical grade. The beads were washed with sterile, double distilled water until the conductivity was less than 0.3 μS (Hanna Instruments, USA). The surface of the beads was modified by incubation in 50% v/v CrSO4 (Merck, Germany) for 5 min. The beads were then washed repeatedly until the pH was identical to that of the distilled water used for the rinsing and the conductivity was less than 0.3 μS (Hanna Instruments, USA). The microtiterplates or syringes were packed with beads and sterilized either by autoclaving or gamma-irradiation.

Blood culture techniques: In all experiments, containers (microtiterplates or 60 ml syringes) packed with beads were filled with freshly drawn human whole blood from healthy, male or female donors, between 20 and 50 years old, without anti-coagulants unless mentioned otherwise. Whole blood cultures were established under sterile, laminar flow conditions (Kendro, Germany). Incubation was carried out aseptically at 37° C., 5% CO2 (Kendro, Germany) for 24 h intervals. After incubation, serum was retrieved and centrifuged (3500 rpm, 10 min., Megafuge, Kendro, Germany). From the microtiterplates 200 ml and from the syringes 10 ml serum was retrieved, which corresponds to approximately 20% of the total original blood volume. The serum was stored at −20° C. The serum has been previously shown to contain increased levels of interleukin-1-receptor antagonist (IL-1Ra), as well as increased levels of IL-4 and IL-10 (Meier et al., Inflam Res. 52:1-4 (2003)). The serum is also known commercially as Orthokine®.

The patients were given epidural perineural injections three times once a week. Objective and subjective assessments were made at six times (t1-t6) per patient including visual analog scale (VAS) (Joyce et al., Eur J Clin Pharmacol. 14:415-20 (1975)), Oswestry Pain Questionnaire (Fairbank et al. Spine, 25:2940-2953(2000)), SF-36 (short form health survey) (Ware et al., Med. Care, 30:473-483 (1992)), and standardized clinical examination. 2 ml of Orthokine® was injected. One group was given the ILI-Ra conditioned serum (Orthokine®), another was given 10 mg Triamcinolone, and the last was given 5 mg Triamcinolone. Triamcinolone is a steroid commonly used to treat inflammation, allergies, arthritis, and asthma. It has been shown that Triamcinolone is effective at reducing lumbar radicular pain. (randomized double blind study, Kramer Eur Spine 1997.

### 14.1.2 Results

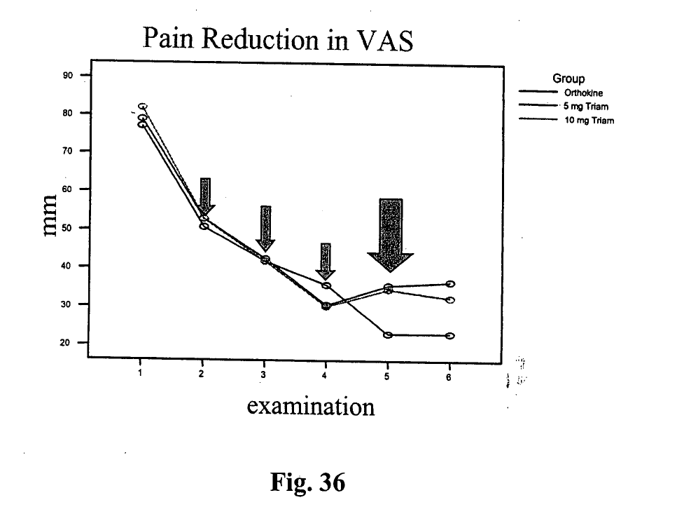

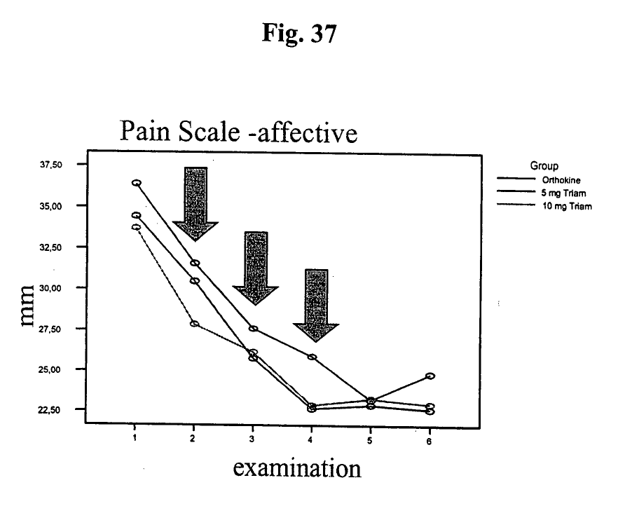

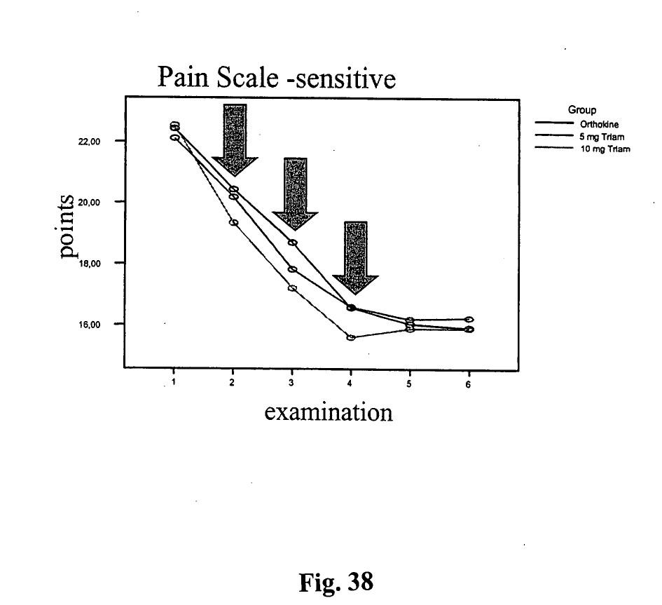

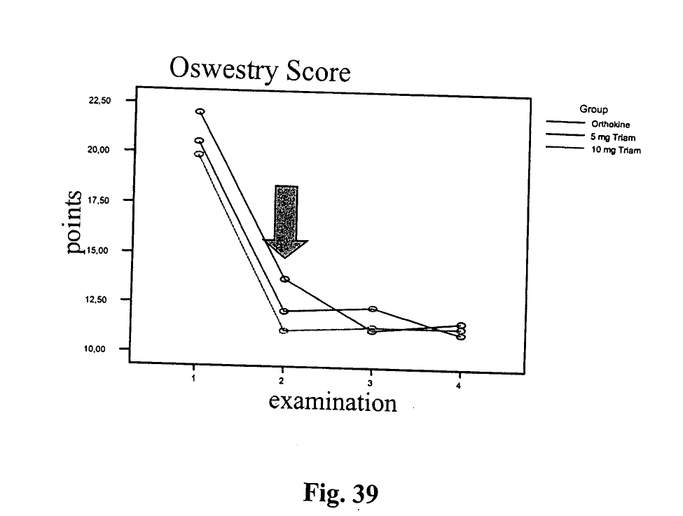

A significant reduction of pain (VAS) occurred after every injection (p<0.01). There was a significant difference between Orthokine® and Triam groups (p<0.001). While pain increases after six weeks (t4) in the Triam groups, reduction persists in the Orthokine group (FIG. 36). There was no significant difference between Triam 5 mg and 10 mg. There was significant reduction between t1 and t4 on the SESaff (Affective Pain Scale), but with no difference between the groups (FIG. 37). There was significant reduction between t1 and t4 on the SESsens (Sensitive Pain Scale), but with no difference between the groups (FIG. 38). There was significant reduction between t1 and t4 on the Oswestry Score, but with no difference between the groups (FIG. 39). No significant adverse effects were observed in any of the three groups.

The results observed in the Orthokine® group is significantly better then the observed results for the low and high Triamcinolone groups.

### 14.2 Orthokine® Serum Contains Exosomes

### 14.2.1 Materials and Methods

An exosome fraction was prepared from a preparation of serum as described in Example 16. The serum was isolated from human blood. Exosomes were enriched from the serum by differential centrifugation, loaded onto a Formvar/carbon coated grid, negatively stained with 10 μl neutral 1% aqueous phosphotungastic acid and viewed using a JEOL-1210 computer controlled high contrast 120 kv transmission electron microscope.

### 14.2.2 Results

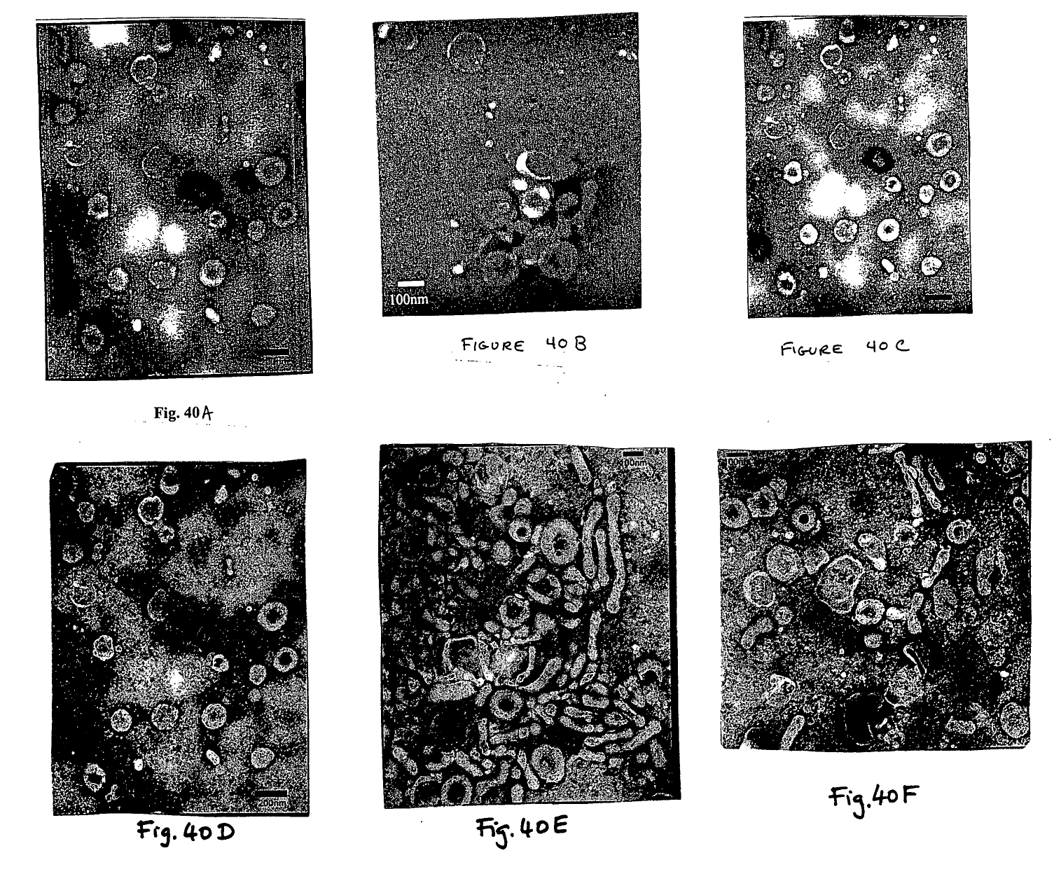

FIG. 40A-C clearly show the presence of exosomes in the enriched Orthokine® serum.

### 14.3 Preparation of Concentrated Exosomes from Orthokine® Serum

Exosomes were produced by incubation of whole blood in specific surface treated syringes (Orthokine® syringes). Cells excreted the vesicles from the endoplasmic reticulum over a time period of 6-24 hrs. This process could be enhanced by addition of 5 μg IL-1ra (IRAP)per ml of whole blood (Example A-D, below). In Examples A-D after incubation the serum was separated from the blood cells by centrifugation at 5000 g for 10 min. The serum then contained varying amounts of exosomes. The concentration of the vesicles was performed by a second centriftigation at 100,000 g for 1 hr. From 20 ml of serum, after centrifugation, an approximately 1 ml volume pellet was withdrawn from the bottom of the centrifuge vial and filtered (through a 0.2 μM filter) into screw cap vials. This 1 ml exosome solution was later injected into patients. Sometimes a higher dose (e.g., five 1 ml aliqouts of concentrated exosomes) were administered as a single injection.

### 14.4 Treatment of Human Subjects with Concentrated Exosomes

### 14.4.1 Example A

STATUS BEFORE EXOSOME TREATMENT: The subject was a male, 22 years old, suffering from juvenile rheumatoid arthritis (type Oligo-II with sacroiliitis, coxitis, gonarthritis, HLA-B 27 positive) for approximately 10 years. He had been treated with MTX 10 mg per week and decortin 5 mg per week, but continued to suffer severe pain and swelling of his right knee; steroid injections of his swollen joints produced no improvement.

Upon examination, before exosome treatment, the subject presented with a blood pressure of 90/60 mm Hg and decreased shoulder movements, with abduction of 50 degrees. Both knees were extremely swollen, with an extension deficit of 10 degrees. X-ray studies of the subject's knees demonstrated effusions but no significant cartilage destruction, and X-ray studies of his hips indicated level IV cartilage destruction with an indication for hip arthroplasty. Blood tests revealed CRP 9.9 mg/dl ; BSR 59; RF,CCP-AK and ANA negative. Anemia of 11.3, and thrombocytes 511 000 μl.

In summary, the subject presented with clinical indicie of severe juvenile arthritis which was refractory to conventional therapy.

EXOSOME TREATMENT. Exosomes were prepared from autologous peripheral blood as described above in 14.3. As the right knee was very painful, swollen and the primary affected joint, it was decided to inject the right knee joint. The therapeutic goal was to decrease swelling and pain in the right knee and to have a therapeutic effect in the other joints affected. Intraarticular injection of 1 ml of concentrated exosomes prepared from 20 ml of conditioned serum was performed without complications into the right knee.

Four weeks after injection there was a 100% decrease of pain in both knees, the WOMAC score was significantly decreased in both knees, and there was a 100 percent decrease of pain in both shoulders. The CRP level decreased to 6 mg/dl, and swelling of the right knee decreased by 3 cm compared to pretreatment value. The patient was extremely pleased with the outcome.

Nine weeks after the first injection of concentrated exosomes, a second injection of 1 ml of concentrated exosomes prepared from 20 ml of conditioned serum was administered to the right knee, and 1 ml of concentrated exosomes prepared from 20 ml of conditioned serum was injected intraarticularly into each of the left shoulder and left knee The right knee was found to show 100 percent improvement, the left knee showed 70 percent improvement, and the shoulders showed 80 percent improvement.

Four weeks after the second injection (13 weeks after the first injection), further concentrated exosome treatment was administered.

Following treatment, the patient's juvenile rheumatoid arthritis was in remission, as confirmed by the evaluation of an independent rheumatologist. Thus, concentrated exosome therapy appeared to be more effective than MTX and steroids in producing amelioration of the disease process.

### 14.4.2 Example B

STATUS BEFORE EXOSOME TREATMENT: The subject was a 65 year old male, suffering from seropositive Rheumatoid Arthritis for 10 years. His mother was also afflicted with the disease. The subject complained of severe knee pain and exhibited cartilage degradation grade III-IV after Kellgreen. Examination revealed painful hand joints at MCP, painful decrease of shoulder movement, effusion over the left knee, and decreased hip inner rotation on both sides. Blood tests demonstrated SR 28/65; CRP 0.8 mg/ml; RF 211 U/ml; ANA negative, leucocytes 7900, thrombocytes 353 000. X-ray studies revealed RA grade II of hands, and knees grade III-IV. He had been treated with gold therapy, which was continued during exosome therapy.

EXOSOME TREATMENT: Exosomes were prepared from autologous peripheral blood as set forth in 14.3. Intraarticular injection of both knees, each injected with 1 ml of concentrated exosomes prepared from 20 ml of conditioned serum, was uncomplicated without adverse side effects.

4 weeks after injection, the patient was reexamined and found to have tolerated treatment very well in the whole observation period. He indicated an improvement of symptoms of 98% compared to pretreatment values. The WOMAC score showed highly significant better outcome measures (50-80%). Swelling was completely absent compared to pretreatment values with decreased diameter of joint of −2 cm after exosome treatment.

4 weeks later, there was a recurrence of pain of around 50% improvement compared to pre exosome status in the left knee and around 30% in the right knee. A second intraarticular injection of 1 ml of concentrated exosomes prepared from 20 ml of conditioned serum into each of both knees was carried out. Two weeks later, a third exosome injection of 1 ml of concentrated exosomes prepared from 20 ml of conditioned serum into each of both knees was carried out, with an improvement of knees at this time of 70% in both knees compared to pain status concentrated exosome treatment.

Two months later the patient indicated an improvement of pain of 80% in both knees compared to pre-exosome therapy status. The WOMAC scores continued to be significantly improved relative to pretreatment values.

### 14.4.3 Example C

STATUS BEFORE EXOSOME TREATMENT: The patient was a 20 year old female with a juvenile Morbus Still (known for 8 years) with contractures of the knees, bilateral hip endoprotheses, bilateral lateral knee cartilage degradation, osteoporosis and perimyocarditis. She had received long time steroid therapy and her disease was considered refractory to therapies such as anti TNF, steroids, MTX, and combinations thereof. Blood tests revealed CRP 20 mg/ml, BSR 98, and evidence of severe immune defect in all other parameters; Leucocytois of 22900/nl, thrombocytosis of 560 000/nl. The subject elected to discontinue her previous therapies due to lack of effectiveness, and for half a year the blood parameters remained basically unchanged. CRP values were between 12.3 and 11.5 mg/ml with a leucocytosis of 22 300/nl. During concentrated exosome therapy, no other chemical therapeutic modalities were administered.

EXOSOME TREATMENT. Exosomes were prepared from autologous peripheral blood as set forth in 14.3. lntraarticular injection of 1 ml of concentrated exosomes prepared from 20 ml of conditioned serum into each of both knees was carried out without complications.

Two weeks later, 1 ml of concentrated exosomes prepared from 20 ml of conditioned serum was injected, intraarticularly, into each of both shoulders without complications.

Four weeks after the first injection, the patient indicated improvement of pain in her left shoulder by 100%, her right shoulder by 70%, and both knees by 100% compared to pretreatment values.

Eight weeks after the first injection, 1 ml of concentrated exosomes prepared from 20 ml of conditioned serum was injected intraarticularly into the right shoulder.

Fifteen weeks after the first injection, CRP had decreased from 12.3 mg/ml to 8.3 mg/ml, and the subject reported that her knee was 80% better and her shoulders were around 60% better.

Sixteen weeks after the first injection, concentrated exosomes were injected intraarticularly into both knees. The subject reported 80% improvement in knees and shoulders, and stated that there was more than 50% improvement in other joints as well. The patient indicated that she would rather not resume conventional therapy in view of her improvement after concentrated exosome treatment.

Seven months after the first injection of exosomes, the patient experienced some decline of the beneficial clinical effects, and it was decided to increase the dose of exosomes by a factor of 6. At this time point, CRP was at 8.6 mg/ml and leucocytes at 22 500/nl. A high dose of exosomes, namely 5 ml of concentrated exosomes prepared from 100 ml of conditioned serum, was intraarticularly injected into each of both shoulders.

One week later, the patient was found to exhibit 80% improvement relative to pre-high dose exosome injection, with improvement of shoulder movements around 30 degrees in abduction; and knees and hands 50% improved. The WOMAC score improved dramatically by more than 50% for the knees, even though high dose injection had been administered to the shoulders. CRP decreased to 7.2 mg/dl, and leucocytes to 18 800/ml.

### 14.4.4 Example D

STATUS BEFORE EXOSOME TREATMENT: The patient was 49 years old, with an 18 year history of hay fever and a proven allergy to grass and pollen.

EXOSOME TREATMENT. Exosomes were prepared from autologous peripheral blood as set forth in 14.3. 5 ml of concentrated exosomes prepared from 100 ml of conditioned serum were administered by subcutaneous injection, and, 2 weeks later, there was a marked decrease in the symptoms of hay fever, such as sneezing and inflammation of the eyes and nose. Sneezing frequency decreased from 124 sneezes per day to 0 per day. The effect persisted through the hay fever season. Treatment did not produce any adverse side effects.

### 14.5 Further Clinical Studies Using Orthokine® Serum

Serum was prepared as set forth in section 14.1, above, and in Meier et al., Inflam Res. 52:1-4 (2003). In these patient series no additional external IL-Ira was used and no 100 000 g centrifugation step to concentrate exosomes was performed. Therefore, in addition to a less concentrated population of exosomes, autologous cytokines and growth factors are present.

56 Patients with rheumatoid arthritis and joint changes of the hands, either previously treated with conventional therapy or previously untreated, were treated with 4-6 injections of Orthokine® serum (2 ml/injection) twice a week into various affected RA joints. Clinical follow up lasted up to 4 years. The average pain improvement was 65.31% SE 7.00 after 3 months compared to pretreatment values. The responder rate was 68.8%, indicating that these patients had at least a 50% improvement of pain in the affected joint compared to the preinjection values after 3 months. On average, these values declined as a function of time after treatment. An additional co-injection of steroids into the affected joint had no beneficial, statistically significant effect on the 3 months results compared to the Orthokine® serum injection. No side effects occurred.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the contents of which are hereby incorporated by reference in their entireties.

What is claimed is:

1. A method of inhibiting an immune response in a subject in need of such treatment, comprising administering, to the subject, an effective amount of exosomes prepared from a culture of antigen presenting cells.

2. The method of claim 1, wherein the antigen presenting cells are exposed to an enhancing agent in culture.

3. The method of claim 1, wherein the antigen presenting cells are genetically engineered to express an enhancing agent.

4. The method of claim 1, 2 or 3, wherein the antigen presenting cells are dendritic cells.

5. The method of claim 2 or 3, wherein the enhancing agent is IL-4.

6. The method of claim 4, wherein the enhancing agent is IL-4.

7. The method of claim 2 or 3, wherein the enhancing agent is IL-10.

8. The method of claim 4, wherein the enhancing agent is IL-10.

9. The method of claim 3, wherein the enhancing agent is FasL.

10. The method of claim 4, wherein the enhancing agent is FasL.

11. The method of claim 1, wherein the immune response to be inhibited is manifested as arthritis.

12. The method of claim 11, wherein the arthritis is rheumatoid arthritis.

13. The method of claim 1, wherein the immune response to be inhibited is inflammation associated with a wound.

14. The method of claim 1, wherein the immune response to be inhibited is manifested as allergies.

15. The method of claim 1, wherein the immune response to be inhibited is manifested as asthma.

16. The method of claim 1, wherein the immune response to be inhibited is manifested as diabetes mellitus type I.

17. The method of claim 1, wherein the immune response to be inhibited is an autoimmune response.

18. The method of claim 17, wherein the autoimmune response is manifested as an autoimmune disease selected from the group consisting of rheumatoid arthritis, juvenile rheumatoid arthritis, systemic lupus erythematoisis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo.

19. A method of inhibiting an immune response in a subject in need of such treatment, comprising administering, to the subject, an effective amount of concentrated exosomes prepared from serum.

20. The method of claim 19, wherein the serum is prepared from peripheral blood which had been incubated in the presence of glass beads.

21. The method of claim 20, wherein an enhancing agent is added to the peripheral blood prior to incubation in the presence of glass beads.

22. The method of claim 21, wherein the enhancing agent is interleukin 1 receptor antagonist protein.

23. The method of claim 19, wherein the immune response to be inhibited is manifested as arthritis.

24. The method of claim 23, wherein the arthritis is rheumatoid arthritis.

25. The method of claim 19, wherein the immune response to be inhibited is inflammation associated with a wound.

26. The method of claim 19, wherein the immune response to be inhibited is manifested as allergies.

27. The method of claim 19, wherein the immune response to be inhibited is manifested as asthma.

28. The method of claim 19, wherein the immune response to be inhibited is manifested as diabetes mellitus type I.

29. The method of claim 19, wherein the immune response to be inhibited is an autoimmune response.

30. The method of claim 19, wherein the autoimmune response is manifested as an autoimmune disease selected from the group consisting of rheumatoid arthritis, juvenile rheumatoid arthritis, systemic lupus erythematoisis, scleroderma, Sjogren's syndrome, diabetes mellitus type I, Wegener's granulomatosis, multiple sclerosis, Crohn's disease, psoriasis, Graves' disease, celiac sprue, alopecia areata, central nervous system vasculitis, Hashimoto's thyroiditis, myasthenia gravis, Goodpasture's syndrome, autoimmune hemolytic anemia, Guillan-Barre syndrome, polyarteritis nodosa, idiopathic thrombocytic purpura, giant cell arteritis, primary biliary cirrhosis, Addison's disease, ankylosing spondylitis, Reiter's syndrome, Takayazu's arteritis, and vitiligo.

31. A composition comprising exosomes prepared by culturing antigen presenting cells in the presence of an enhancing agent and collecting exosomes from the culture supernatant.

32. The composition of claim 31, wherein the enhancing agent is a cytokine.

33. The composition of claim 32, wherein the enhancing agent is selected from the group consisting of interleukin 4 and interleukin 10.

34. The composition of claim 31, wherein the enhancing agent is a cytokine inhibitor.

35. The composition of claim 34, wherein the enhancing agent is interleukin-1 receptor antagonist protein.

36. The composition of claim 31, wherein the enhancing agent is a NFκB inhibitor.

37. A composition comprising exosomes prepared by culturing antigen presenting cells which have been engineered to express an enhancing agent and collecting exosomes from the culture supernatant.

38. The composition of claim 37, wherein the enhancing agent is a cytokine.

39. The composition of claim 38, wherein the enhancing agent is selected from the group consisting of interleukin 4 and interleukin 10.

40. The composition of claim 37, wherein the enhancing agent is a cytokine inhibitor.

41. The composition of claim 40, wherein the enhancing agent is interleukin-1 receptor antagonist protein.

42. The composition of claim 37, wherein the enhancing agent is FasL.

43. A composition comprising exosomes prepared by collecting exosomes from serum.

44. A composition comprising exosomes prepared by collecting exosomes from serum prepared from a peripheral blood sample that has been incubated with glass beads.

45. The composition of claim 44, wherein an enhancing agent is added to the peripheral blood sample which is incubated.

46. The composition of claim 45, wherein the enhancing agent is interleukin 1 receptor antagonist protein.
